(19) United States
(12) Patent Application Publication
(10) Pub. No.: US 2021/0327582 A1
(43) Pub. Date: Oct. 21, 2021

(54) METHOD AND SYSTEM FOR IMPROVING THE HEALTH OF USERS THROUGH ENGAGEMENT, MONITORING, ANALYTICS, AND CARE MANAGEMENT

(71) Applicant: Asha AI, Inc., Redmond, WA (US)

(72) Inventor: Rashmi Joshi, Redmond, WA (US)

(21) Appl. No.: 17/231,871

(22) Filed: Apr. 15, 2021

Related U.S. Application Data
(60) Provisional application No. 63/010,584, filed on Apr. 15, 2020.

(51) Int. Cl. G16H 50/20; G16H 10/60; G16H 40/67; G16H 40/20; G06Q 50/00; G06Q 10/10; G06N 20/00; A63F 13/67

(52) U.S. Cl. CPC G16H 50/20; G16H 10/60; G16H 40/67; G16H 40/20; G16H 15/00; G06Q 10/1095; G06N 20/00; A63F 13/67; G06Q 50/01

(57) ABSTRACT

Systems and methods applicable, for instance, to using engagement, monitoring, analytics, and care management to improve the health of users. Various software modules can be provided. Further provided can be various machine learning models.

### CROSS-REFERENCE TO RELATED APPLICATIONS

This application claims priority to U.S. Provisional Patent Application Ser. No. 63/010584, filed on Apr. 15, 2020, the contents of which are incorporated herein by reference in their entirety and for all purposes.

### FIELD

The present technology relates to the field of improving the health of users. More particularly, the present technology relates to techniques for improving the health of users via engagement, monitoring, analytics, and care management.

### BACKGROUND

Many individuals in the US suffer from conditions that require regular monitoring. For example, approximately 15.3 million Americans in the 18-64 age group have been diagnosed with diabetes. Further, as individuals age they become more susceptible to an increasing number of health conditions that require regular monitoring. As of 2020, approximately 17% of the US population is 65 years old or older. This number is expected to rise to nearly 21% by 2030.

Approximately 95% of elderly people in US live independently. This is not surprising, given the high cost of professional caregiving services. While living alone preserves the independence of the elderly, it stifles their access to family members who can help them manage their conditions more effectively. Separation from family members can also result in feelings of isolation, helplessness and depression, which can further exacerbate their conditions and result in a decline in their quality of life. Furthermore, such elderly individuals often suffer from multiple acute and chronic conditions, such as infections, high blood pressure, irregular heartbeats, hypoglycemia or hyperglycemia, and others. And, having multiple chronic conditions is not a fate limited to the elderly. Instead, four of every ten adults in the US have two or more chronic diseases.

Healthcare solutions that are on the market today are oriented to tech-savvy people. As such, average, non-tech-savvy people of all ages tend to find these solutions unappealing or impossible to use. Where a person has accessibility issues (e.g., vision, hearing, mobility, and/or fine motor skill difficulties), using these tech-savvy-oriented healthcare solutions can become even more difficult. Many elderly individuals suffer from these accessibility issues. Absent from existing healthcare solutions are, for instance, user interfaces that would extend access to a non-tech-savvy and/or accessibility-limited users.

Additionally, existing healthcare solutions tend to be limited in scope. For example, many existing healthcare solutions do little more than allowing a user to track sensor-obtained information (e.g., heart rate or ECG), or store limited health information (e.g., height, weight, blood pressure, and sleep cycles). Absent from these existing healthcare solutions is, for example, leveraging health data to provide meaningful insights.

Further still, existing healthcare solutions fail to provide functionality such as connecting users with family members or with care team members (e.g., physicians). As such, these healthcare solutions do not, as just one example, help address the isolation and separation that plagues elderly individuals and others who live alone. Further, although providing connection to food delivery services and laundry services could prove useful to elderly individuals and others, existing healthcare solutions fail to help secure these services for users.

As such, there is call for technologies which are applicable to overcoming the aforementioned deficiencies of existing healthcare solutions.

### BRIEF DESCRIPTION OF THE DRAWINGS

FIG. 1A shows example software modules, according to various embodiments.

FIG. 1B shows an example high-level architecture diagram, according to various embodiments.

FIG. 1C shows an example connectivity/data access diagram, according to various embodiments.

FIG. 1D shows an example data storage/access diagram, according to various embodiments.

FIG. 2A shows an example of alert/notification/reminder functionality, according to various embodiments.

FIG. 2B shows an additional example of alert/notification/reminder functionality, according to various embodiments.

FIG. 2C shows a further example of alert/notification/reminder functionality, according to various embodiments.

FIG. 3A shows an example of machine learning-based functionality, according to various embodiments.

FIG. 3B shows an additional example of machine learning-based functionality, according to various embodiments.

FIG. 3C shows an example personal health record element, according to various embodiments.

FIG. 4 shows a further example of machine learning-based functionality, according to various embodiments.

FIG. 5 shows yet another example of machine learning-based functionality, according to various embodiments.

FIG. 6 shows an additional example of machine learning-based functionality, according to various embodiments.

FIG. 7 shows a further example of machine learning-based functionality, according to various embodiments

FIG. 8 shows another example of machine learning-based functionality, according to various embodiments.

FIG. 9A shows an example of machine learning-based functionality, according to various embodiments.

FIG. 9B shows an additional example of machine learning-based functionality, according to various embodiments.

FIG. 9C shows a further example of machine learning-based functionality, according to various embodiments.

FIG. 10 shows an example of call functionality, according to various embodiments.

FIG. 11A shows an example care directory access screenshot, according to various embodiments.

FIG. 11B shows an additional example care directory access screenshot, according to various embodiments.

FIG. 12A shows an example conversation transcript screenshot, according to various embodiments.

FIG. 12B shows an example metadata screenshot, according to various embodiments.

FIG. 13 shows an additional example of call functionality, according to various embodiments.

FIG. 14A shows an example of interfacing with a pharmacy.

FIG. 14B shows an additional example of interfacing with a pharmacy.

FIG. 15 shows an example of game functionality, according to various embodiments.

FIG. 16 shows an example computer, according to various embodiments.

### DETAILED DESCRIPTION

### General Operation

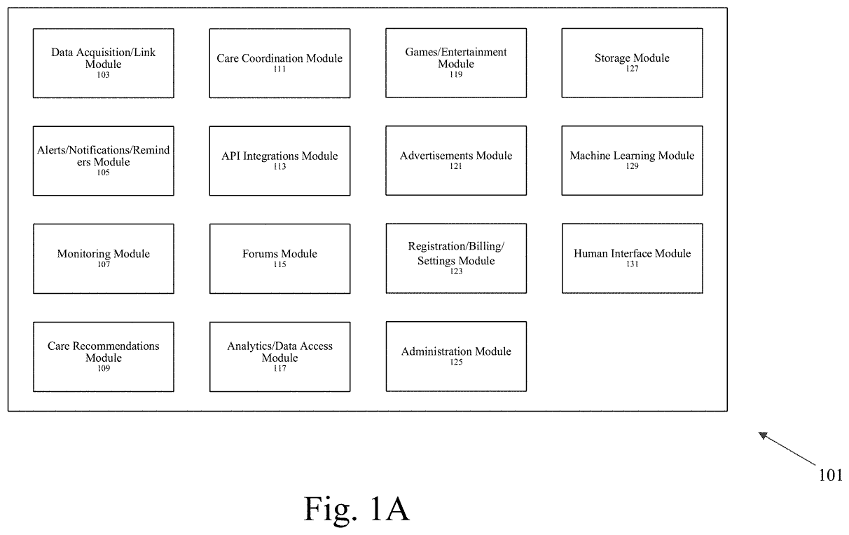

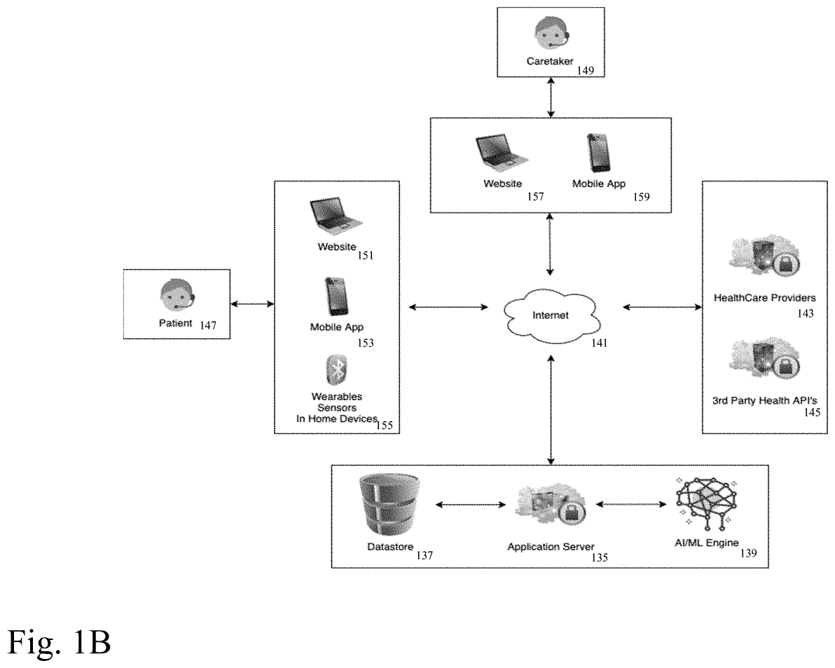

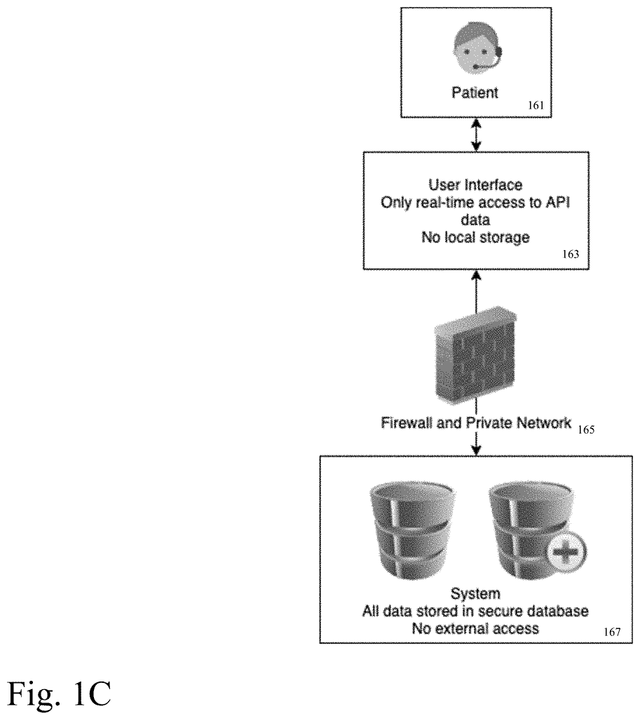

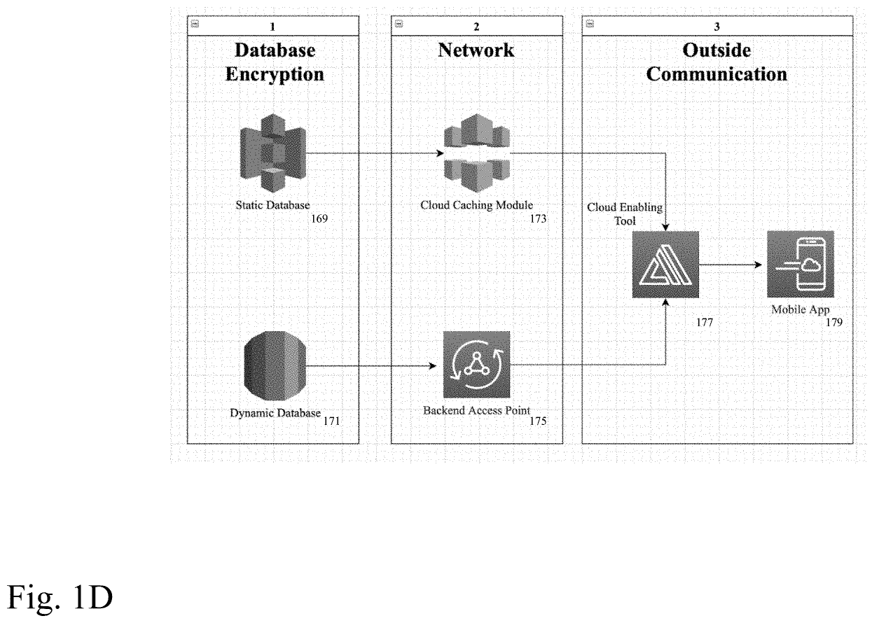

According to various embodiments, there are provided systems and methods for improving the health of users through engagement, monitoring, analytics, and care management. As an example, such systems and methods can, as depicted by FIG. 1A, be implemented via system 101 having software modules 103-131. The data acquisition/link module 103 can perform operations including accessing/receiving medical record data. The data acquisition/link module 103 can also perform operations including accessing/receiving internet of things (IoT)/health-monitoring device data. The alerts/notifications/reminders module 105 can perform operations including communicating various information to family members, care team members (e.g., physicians and pharmacies), and a monitored user. In particular, the alerts/notifications/reminders module 105 can inform various of such individuals of, as just some examples, upcoming medicine dosings, missed medicine dosings, emergent/potentially emergent situations (e.g., falls and cardiac irregularities), and care recommendations, and/or predicted conditions/health statuses. Then, the monitoring module 107 can perform operations including monitoring for the noted emergent/potentially emergent situations. A monitored user can be a patient who is being monitored by the system. The monitored user can be an elderly individual, an individual with a diagnosed health condition, or an individual managing a chronic condition (e.g., diabetes or Parkinson's disease), as just some examples. Family members can include, as just some examples, relatives, friends, caregivers, and legal guardians of the monitored user. Such individuals can be involved in managing care for the monitored user. It is noted that family members are not limited to blood relatives of the monitored user. Care team members can include, as just some examples, physicians, clinicians, pharmacists, nurses, and caregivers of the monitored individual. Family members and care team members can, from one point of view, both be considered to be monitoring users. Further, although the terms “family member” and “care team member” are used at various locations hereinthroughout, it is noted that various actions and functionality discussed in terms of a family member can apply to a care team member. Likewise, various actions and functionality discussed in terms of a care team member can apply to a family member.

The care recommendations module 109 can perform operations including providing the care recommendations, and/or predicted conditions/health statuses. As an example, one or more machine learning models (MLMs) can be used by the care recommendations module 109 in performing such provision. The care coordination module 111 can perform operations including aiding in the coordination of care for the monitored user. As just one example, the care coordination module 111 can facilitate communications between the monitored user, family, and care team members. Further, the Application Programming Interface (API) integrations module 113 can perform operations including connecting the system with 3rd party services and devices. In this way the API integrations module can, for instance, assist in securing support services (e.g., food delivery and laundry services).

The forums module 115 can perform operations including hosting community forums which allow users to discuss various issues (e.g., eldercare issues) with one another. The analytics/data access module 117 can perform operations including generating data reports. The generated data reports can include reports that regard the monitored individual. The generated data reports can also include reports that regard operational performance of the system. The analytics/data access module 117 can also perform data analysis operations, and logging and auditing operations (e.g., tracking data accesses, medical interventions, medical outcomes, care recommendations, and predicted conditions/health statuses).

The games/entertainment module 119 can perform operations including providing health, fitness, and wellness games (e.g., brain health, exercise, and/or dexterity games. The advertisements module 121 can perform operations including selecting advertisements to be displayed to the monitored user. Further, the registration/billing/settings module 123 can perform operations including registering a new monitored user with the system and handling billing of fees incurred through usage of the system. The registration/billing/settings module 123 can also perform operations including allowing for the selection/viewing of various settings relating to usage of the system. Then, the administration module 125 can perform operations including allowing system administrators to perform various administrative tasks relating to the system.

The storage module 127 can perform operations including handling the storage, retrieval, and/or encryption of various data received and generated by the system. As an example, the storage module 127 can interface with one or more databases. Further, the storage module 127 can perform data governance operations to ensure the quality, integrity, security, and usability of data utilized by the system. The machine learning module 129 can perform operations including providing access to MLMs used by the system. For instance, the care recommendations module 109 can use one or more MLMs provided by the machine learning module 129 when providing the noted care recommendations. Family, care team members, and the monitored user can interface with the system in various ways, such as via a mobile app (e.g., via an iOS, Android, or Jitterbug app) and via a virtual assistant capability (e.g., via an Amazon Alexa skill, a Google Assistant action, or a Siri shortcut). Where, for example, an Amazon Alexa skill is used the system can utilize the voice functionality of Alexa along with backend services provided by Amazon. Users can engage with the skill using an Amazon Echo device. The human interface module 131 can perform operations including interfacing with such apps and virtual assistant capabilities. Although mobile apps and virtual assistant capabilities are referenced hereinthroughout to facilitate discussion, it is noted that, in various embodiments, other interfaces can be employed instead of or in addition to such apps and capabilities, and that the system can be considered to be device agnostic. As just some examples, such interfaces can include web interfaces, IoT interfaces, smartwatch interfaces (e.g., smartwatch apps), virtual reality (VR) interfaces, and augmented reality (AR) interfaces,

Implementation of the software modules 103-131 can include utilizing one or more frameworks, application program interfaces (APIs), and/or web services. As just some examples the frameworks/APIs can be Apple or Java frameworks/APIs, and the web services can be Amazon Web Services (AWS) web services. The software modules 103-131 can, as just some examples, communicate with one another (and/or with other software modules) via one or more HTTP APIs, and/or via interprocess communication functionality offered by the runtime environment and/or operating system running the software modules 103-131. Also, the software modules 103-131 can, in various embodiments, communicate with web services, the mobile app, the virtual assistant capability, IoT devices, and/or other entities via one or more HTTP APIs. As an example, such a Hypertext Transfer Protocol (HTTP) API can utilize passed JavaScript Object Notation (JSON) data structures. Further still, the software modules 103-131 can interface with one or more databases or other storage locations. As some examples, the software modules 103-131 can run on one or more servers, and/or be deployed via Amazon Elastic Computing Cloud (EC2). Various aspects will now be discussed in greater detail.

Turning to FIG. 1B, shown is an example high-level architecture diagram, according to various embodiments. As depicted by FIG. 1B, the system discussed herein can include an application server 135 which can run one or more of the discussed software modules. The system can also include a datastore 137, which can interface with the storage module 127. Then, as noted, the machine learning module 129 can have access to various MLMs. These MLMs are depicted in FIG. 1B as AI/ML engine 139.

With further reference to FIG. 1B, the system can interact with various devices and individuals via the internet 141, SMS (not shown), and/or Bluetooth (e.g., for connection to a web or mobile app; not shown). As just some examples, the system can utilize Bluetooth hardware of an IoT device (e.g., an Amazon Echo) or a PC for the Bluetooth connection. For example, the system can use the internet to access healthcare provider organizations 143 and third-party health APIs 145. Also via the internet the system can interact with the monitored user 147 (labeled “Patient” in FIG. 1B) and with one or more care team members 149 (labeled “Caretaker” in FIG. 1B). The system can interact with/receive data from the monitored user via a website 151, a mobile app 153, a virtual assistant capability (not shown), text messaging (not shown), postal mail (not shown) and IoT/health-monitoring devices 155 (labeled “Wearable Sensors, In Home Devices” in FIG. 1B). Then, the system can interact with the care team member via a website 157, a mobile app 159, a virtual assistant capability (not shown), and/or an IoT device (not shown).

Turning to FIG. 1C, shown is an example connectivity/data access diagram, according to various embodiments. As depicted by FIG. 1C, the monitored user 161 (labeled as “Patient” in FIG. 1C) can access the system via the mobile app or the virtual assistant capability. As indicated by element 163 of FIG. 1C, the app or virtual assistant capability (labeled “User Interface” in element 163 of FIG. 1C) can be implemented such that the access utilizes an API (e.g., an HTTP API), and such that no data (or no sensitive data) is stored at the app or virtual assistant capability. Further, as depicted by element 165, the app or virtual assistant capability can access the system through a firewall and/or via a Virtual Private Network (VPN). Then, as depicted by element 167, storage can be implemented such that all data (or all sensitive data) is stored in a secure database, and such that direct external access to the data is disallowed.

In various embodiments, data utilized by the system can be segregated into static data and dynamic data. The static data can include data which is unlikely to change (or unlikely to change frequently). As just some examples, static data and include name, age, and historical conditions. The dynamic data can include data which has a tendency to change. As just some examples, the dynamic data can include current symptoms and IoT/health-monitoring device data. Turning to FIG. 1D, shown is an example data storage/access diagram, according to various embodiments. As depicted by FIG. 1D, the static data can be stored in a static database 169. As just an example, the static database can be implemented via Amazon Simple Storage Service (S3). Further, the dynamic data can be stored in a dynamic database 171. As just an example, the dynamic database can be implemented via Amazon Relational Database Service (RDS). As also depicted by FIG. 1D, access to the static database can utilize a cloud caching module 173. The cloud caching module can act to speed up distribution of data via edge servers. As just an example, the cloud caching module can be implemented via Amazon CloudFront.

As also depicted by FIG. 1D, access to the dynamic database can utilize a backend access point 175. The backend access point can provide a serverless GraphQL service which facilitates queries and other data operations. As just an example, the backend access point can be implemented via AWS AppSync. Further still, a cloud enabling tool 177 can interface with the cloud caching module and the backend access point. The cloud enabling tool can provide a serverless framework which facilitates interface with the mobile app 179 and the virtual assistant capability. As just an example, the cloud enabling tool can be implemented via AWS Amplify. It is noted that, in various embodiments, other than a serverless framework can be used. It also is noted that, in various embodiments, data is not segregated into static data and dynamic data.

### Alert, Notification and Reminder Operations

As referenced, the system can provide various information to family, care team members, and the monitored user. The system can communicate this information in the form of alerts, notifications, and reminders. In various embodiments, the system can perform such operations via the alerts/notifications/reminders module 105.

As just some examples, the alerts can regard emergent/potentially emergent situations (e.g., falls and cardiac irregularities), missed medications, and missed medical appointments. The notifications can, as just some examples, regard care recommendations, data reports, care coordination reports, and prescription refill statuses. Then, as just some examples, the reminders can regard scheduled medication dosings, upcoming health or wellness appointments, and upcoming calls with family. The system can provide the alerts, notifications, and reminders via a mobile app and/or virtual assistant capability. In this regard, the system can utilize the human interface module 131. Further, the system can provide the alerts, notifications, and reminders via audio, video, push notification, mobile messaging (e.g., SMS or iMessage), and messaging via IoT devices. In this regard, the system can utilize the care coordination module 111. In various embodiments, an alert, notification, or reminder intended for a first user (e.g., the monitored user) can be shared with a second user (e.g., a family member or a care team member) or a group of users, with the consent of the first user. In providing such sharing consent, the first user can choose to grant the second user (or the group of users) one or more specified privileges with respect to the alert, notification, or reminder. Such privileges can include the ability to view, the ability to edit, and the ability to share with further users the alert, notification, or reminder.

In handling the time-based alerts, notifications, and reminders (e.g., the missed medication alerts and the scheduled medication reminders), the system can, as just an example, use the Google Calendar API to monitor for relevant circumstances (e.g., a medication dosing coming due). As another example, the system can utilize a calendar module of the system. In handling the emergent/potentially emergent alerts, the system can receive indication of relevant circumstances (e.g., a fall) from the monitoring module 107. In handling the care recommendations notifications, the system can receive indication of a new recommendation/prediction from the care recommendations module 109. In handling the data report notifications, the system can receive indication of a new data report from the analytics/data access module 117. Then, in handling the prescription refill status notifications the system can (e.g., via the data acquisition/link module 105) use a pharmacy API (e.g., the Walgreens Pharmacy Prescription API) to monitor prescription refill status. Further, in various embodiments, the system can utilize the storage module 127 in handling alerts, notifications, and reminders.

In this way, the system can provide family, care team members, and the monitored user with customized alerts, notifications, and reminders. Accordingly, for instance, a care team member can be kept informed of the health of each of their monitored user patients, and can receive various pertinent information (e.g., reminders of upcoming medication dosings and health/wellness appointments). It is noted that alerts, notifications, reminders, storage, and other operations can be implemented in a fashion that respects local regulations regarding health data privacy (e.g., Health Insurance Portability and Accountability Act (HIPAA) regulations in the US). In various embodiments, the administration module 125 can act to ensure that the system meets relevant country-specific regulatory compliance requirements. Further, the system can learn to provide alerts, notifications, and reminders in a fashion that best achieves adherence and safety for the monitored user. For example, the system can learn one or more of: a) when to provide alerts/notifications/reminders; b) what content to include in alerts/notifications/reminders; and c) which type of communication (e.g., alert, notification, or reminder) is most effective for a given circumstance. In performing such learning, the system can consider factors including but not limited to: a) engagement with the alerts/notifications/reminders; b) health data (e.g., vital signs and health record data) of the monitored user; and c) user feedback. Also in performing such learning, the system can utilize one or more MLMs provided by the machine learning module 129.

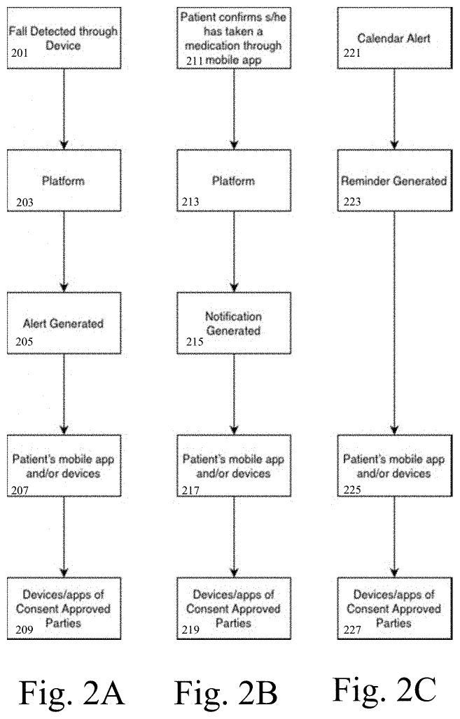

FIGS. 2A-2C show three examples of alert/notification/reminder functionality. Turning to FIG. 2A, at step 201, a fall of the monitored user can be detected by a device. For instance, the device can be a smartwatch worn by the monitored user, and the smartwatch can include an accelerometer. The monitoring module 107 can use the acquisition/link module 103 to communicate with the smartwatch. In this way, the monitoring module 107 can monitor for a fall of the monitored user by looking for accelerometer output which is indicative of a fall (or the smartwatch outputting an indication that its user has fallen). At step 203, the monitoring module 107 can determine that the monitored user has fallen. Subsequently, the monitoring module 107 can provide indication of such to the alerts/notifications/reminders module 105. At step 205, the alerts/notifications/reminders module 105 can generate an alert regarding the fall. At step 207, the alert can, using the human interface module 131, be provided to a mobile app and/or virtual assistant capability of the monitored user. Likewise, at step 209 the human interface module 131 can be used to provide the alert to mobile apps and/or virtual assistant capabilities of other users (e.g., family members and/or care team members) for whom the monitored user has granted consent to share alerts.

Turning to FIG. 2B, at step 211 the human interface module 131 can receive, via the mobile app or the virtual assistant capability, confirmation from the monitored user that they have taken medication so as to satisfy a particular scheduled dosing. The confirmation can be provided in response to a query presented by the mobile app or virtual assistant capability. The human interface module 131 can provide indication that the scheduled dosing has been satisfied to the Google Calendar API. At step 213 the monitoring module 107 can, via the Google Calendar API, learn that the scheduled dosing has been satisfied. Subsequently, the monitoring module 107 can provide indication of the satisfaction to the alerts/notifications/reminders module 105. At step 215, the alerts/notifications/reminders module 105 can generate a notification regarding the satisfaction of the scheduled dosing. At step 217, the notification can, using the human interface module 131, be provided to the mobile app and/or virtual assistant capability of the monitored user. Likewise, at step 219 the human interface module 131 can be used to provide the notification to mobile apps and/or virtual assistant capabilities of other users (e.g., family members and/or care team members) for whom the monitored user has granted consent to share notifications. Where the monitored user fails to confirm that they have satisfied the scheduled dosing, various actions can be performed by the system. For example, the system can provide repeated reminders to the monitored user until they confirm that they have satisfied the dosing. The reminders can be repeated with a frequency chosen by the monitored user, The frequency (e.g., every five minutes) can be stored as application settings.

Turning to FIG. 2C, at step 221 the monitoring module 107 can, via the Google Calendar API, learn that an upcoming medicine dosing is due (or near due). The monitoring module 107 can subsequently provide indication of the upcoming dosing to the alerts/notifications/reminders module 105. At step 223, the alerts/notifications/reminders module 105 can generate a reminder regarding the upcoming dosing. Then, at steps 225 and 227 the reminder can, in a fashion analogous to steps 217 and 219, be provided to the monitored user, and to other users who have been granted consent to share reminders.

### Data Acquisition and Linking Operations

As noted, the system can access/receive various data, including medical record data and IoT/health-monitoring device data. In various embodiments, the system can perform such operations via the data acquisition/link module 103.

In accessing/receiving the medical record data (e.g., electronic health records), the data acquisition/link module 103 can use a medical record API (e.g., the Allscripts API or the Veterans Affairs Health API). As further examples, the data acquisition/link module can use one or more of optical character recognition (OCR), NLP, and fuzzy logic in accessing/receiving the medical record data. For example, the OCR, NLP, and/or fuzzy logic can be applied to imaged faxes, pill bottle prescription labels, and/or reimbursement checks/deposits. The imaging of these inputs can be performed via scanner or smartphone camera, as just some examples. In this way, benefits such as being able to utilize various types of medical forms, semi-unstructured medical data, and unstructured medical data can accrue. The OCR, NLP, and fuzzy logic capabilities can be provided by the machine learning module 129, as just an example. Further still, in various embodiments the system can integrate with electronic health records to share patient health data with the care team (e.g., clinicians thereof). Also, the system can import patient health data through this integration using the system's API framework. The system can, in various embodiments, utilize Fast Healthcare Interoperability Resources (FHIR) for achieving interoperability in terms of health data. It is noted that the term “electronic health record” as used hereinthroughout can refer, for example, to an external electronic health record which is accessed by the system.

In accessing/receiving the IoT/health-monitoring device data, the data acquisition/link module 103 can, as one example, use the AWS IoT API, and/or the relevant IoT/health monitoring devices can be Alexa-enabled. As another example, the data acquisition/link module 103 can access/receive the IoT/health-monitoring device data via the mobile app. Here, the relevant devices can connect to a mobile device upon which the app runs, and the app can access data generated by the relevant devices via an API/framework of the mobile device (e.g., Apple HealthKit). The app can then provide the generated data to the data acquisition/link module 103. It is noted that the system can integrate with a wide variety of IoT/health-monitoring devices, such as through API connection or Bluetooth. As just some examples, the system can send health data of the monitored user to these devices, and can intake data from these devices (e.g., data relating to patient health, service offerings, and/or recommendations). Further the system can display the intaken data via the mobile app and the virtual assistant capability.

The IoT/health-monitoring device data received by the data acquisition/link module 103 can, as just some examples, include data regarding heart rate, blood pressure, insulin/blood sugar (e.g., via device optical sensor), sentiment, calories burnt, sleep (e.g., sleep start/end times and sleep regularity data), mobility (e.g., elapsed time spend sitting, standing, walking, and running), and falls (e.g., via device accelerometer).

As to the data regarding sentiment, as just one example the mobile app or virtual assistant capability can pose to the monitored user a question such as “how are you feeling today?.” The reply of the monitored user can be received by the monitoring module 107 via the human interface module 131. For instance, where the virtual assistant capability is an Amazon Alexa skill, words that the monitored user utters in describing how they feel can correspond to an Alexa slot, and the skill can be configured to have a speech-to-text conversion result of the utterance passed to the human interface module 131 (e.g., via an HTTP API thereof). Subsequently, the monitoring module 107 can use one or more MLMs provided by the machine learning module 129 in order to determine the sentiment of the monitored user. As an example, the monitoring module can utilize a recurrent neural network (RNN) which has been trained to take a sentence as input, and to output a predicted sentiment of the sentence. As another example of determining sentiment, the system can receive an image of the monitored individual. The image can be captured via a smartphone and received by the system via the acquisition/link module 103. Subsequently, the monitoring module 107 can use the image in conjunction with one or more MLMs provided by the machine learning module 129 to determine the sentiment of the monitored user. For instance, a convolutional neural network (CNN)-based MLM which has been trained to take an image of an individual as input, and to output a predicted sentiment of the individual can be used. Alternately or additionally, the captured image can be provided (e.g., a third-party API) to third-party visual recognition software that uses photos for sentiment analysis. As yet another example of determining sentiment, the system can receive location data of the monitored individual via the acquisition/link module 103. The location data can be provided by a smartphone, and be GPS-based or Bluetooth beacon-based, as just some examples. Further, such location data can be correlated by the system with various rooms/locations in the living space of the monitored individual. The monitoring module 107 can use the room/location data in conjunction one or more MLMs provided by the machine learning module 129 to determine the sentiment of the monitored user. As an example, the monitoring module 107 can use a multilayer perceptron (MLP)-based classifier which has been trained to take room/location data as input (e.g., an indication of time spent per day in each of multiple rooms/locations), and to output a predicted sentiment. Such an MLP-based classifier can, as just one illustration, output an indication of negative sentiment when provided with input that indicates that a given individual has been spending a large number of hours in the bedroom or bathroom. As an additional example of determining sentiment, the system can survey the monitored user in this regard. As just an example, the system can, via the mobile app, ask the monitored user to select from among one or more emoticons the particular emoticon which best describes how they are feeling.

Subsequent to the receipt of the IoT/health-monitoring device data by the data acquisition/link module 103, the system can perform various operations. For example, the system can use the storage module 127 to store the data. The storage can be in compliance with local regulations regarding health data privacy (e.g., HIPAA). Also, the storage can be in compliance with various health record data interchange formats (e.g., the FHIR format). Moreover, the system can (e.g., via the storage module 127) scrub and/or reformat the data into labels consistent with a personal health record of the monitored user, as needed. Also, the system can in agreement with that which is discussed earlier, provide (e.g., via mobile app and/or virtual assistant capability) the data to the monitored user, and/or to other users (e.g., family members and/or care team members) for whom the monitored user has granted consent to share data.

Also, the system can analyze received linguistic data for keywords. Such linguistic data can include the above-related data regarding sentiment, or other linguistic data received by the system (e.g., linguistic input provided by the monitored user when using the virtual assistant capability). The analysis of the received linguistic data can include extracting keywords from the linguistic data. The keywords can, as just some examples, regard sentiment of the monitored user, and/or be medically related keywords (e.g., keywords indicative of symptoms and conditions). As an example, the keyword extraction can be performed using one or more MLMs of the machine learning module 129. For instance, in this regard the machine learning module 129 can provide an RNN-based MLM which has been trained to extract from an input sentence keywords of the sort noted. As another example, the keyword extraction can be performed using the Amazon Comprehend Medical webservice. Moreover, the system can use the extracted keywords in connection with one or more MLMs provided by the machine learning module 129 to generate care recommendation outputs, and/or predicted condition/health status outputs. Keywords can include both single words (e.g., “pain”) and multiword units (e.g., “chest pain”). Keyword extraction is further discussed hereinbelow.

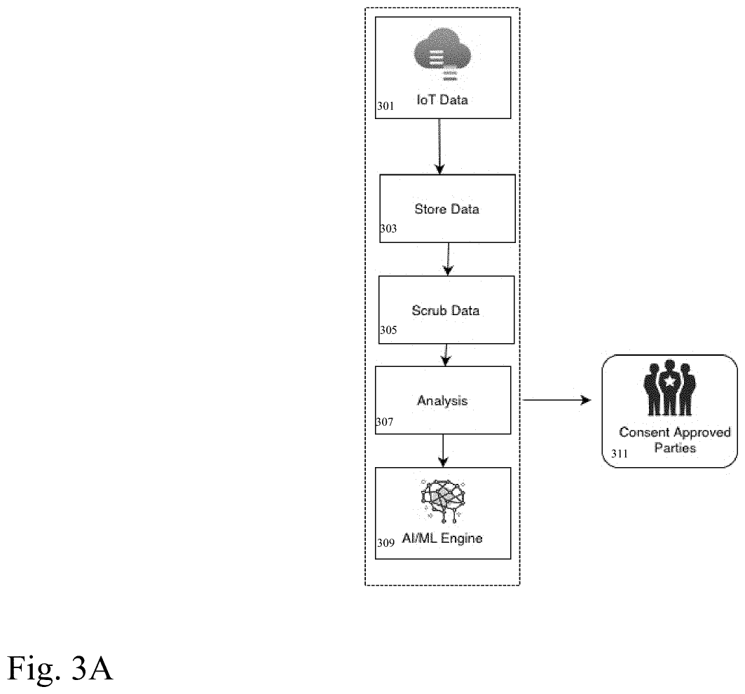

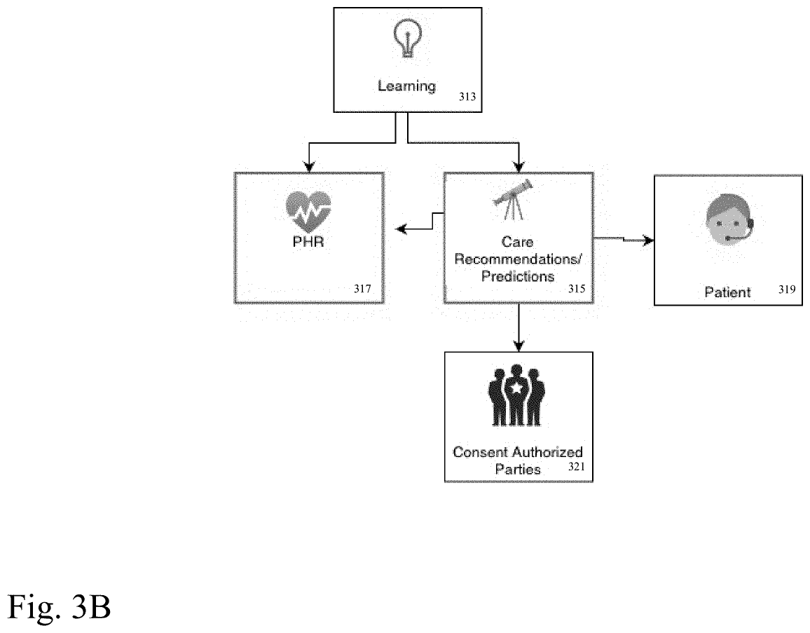

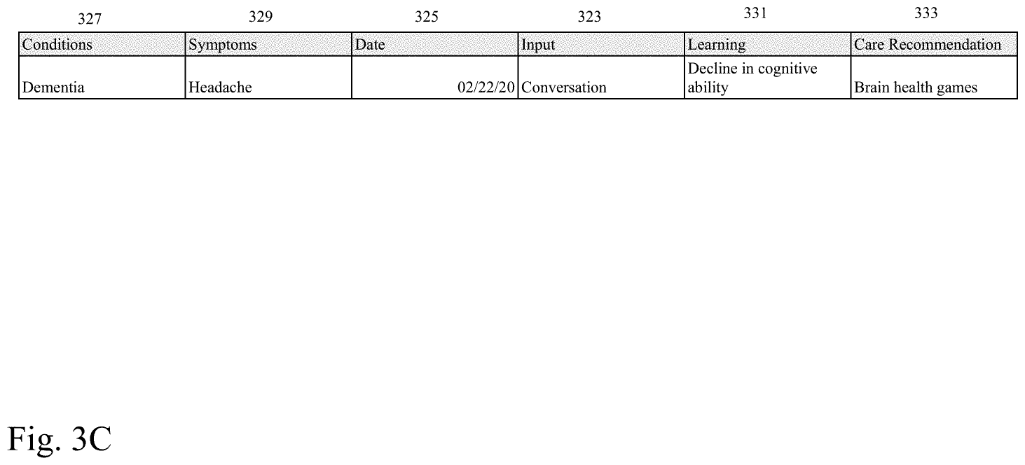

Turning to the example of FIG. 3A, at step 301 the system can access/receive IoT/health-monitoring device data. At step 303 the system can store the data. Then, at step 305, the system can scrub the data. At step 307 the system can analyze received linguistic data for keywords. At step 309 the system can use the extracted keywords in connection with one or more MLMs. Then, at step 311, information can be provided (e.g., via mobile app and/or virtual assistant capability) to the monitored user and/or to consent-granted individuals. As just some examples, the shared information can include the data received from the IoT/health-monitoring devices, and/or results of the use of the MLMs (e.g., the noted care recommendations).

In this way, the system can utilize, for the benefit of the monitored user, data generated by IoT/health-monitoring devices. It is noted that, in general, such devices can include miniaturized devices that collect information (e.g., biometric data, environmental data, and/or information generated by other devices). Also, IoT/health-monitoring devices can have sensors, which can be physically attached to the article/item that they are gathering information from. The IoT/health-monitoring devices can convert this information, using on-board electronics, into digital form that can be transmitted using a tiny radio to the wireless interface of the platform they interface with. The IoT/health-monitoring devices discussed herein can include wearable sensors that are worn by the monitored user monitored by the system. In some embodiments the devices can be off-the-shelf products that can interface with the system as discussed hereinabove (e.g., via AWS IoT), and/or using a wireless interface associated with the system, using standard approaches (e.g., Bluetooth and/or WiFi).

A personal health record for the monitored user can be maintained by the system, for example being stored in a database via the storage module 127. The personal health record can store some or all of the monitored user's patient health data under the monitored user's user account. The personal health record can store data generated by various MLMs of the system and data received by the system from various sources (e.g., electronic health record data and IoT/health-monitoring device data), as just some examples. The personal health record can be considered to be owned by the patient, and can be shared with anyone the patient desires to share it with, including but not limited to family members and care team members (e.g., doctors and pharmacies). Data access to the personal health record can be offered (e.g., via an HTTP API offered by the system) through HIPAA compliant and/or General Data Protection Regulation (GDPR) compliant manners as required by the legal environment of the geographical region within which the patient resides. Furthermore, data can be stored in the personal health record using FHIR methodologies, allowing for benefits including increased interoperability with external healthcare organizations to accrue. In general, all data handling by the system is in compliance with local regulations (e.g., HIPAA for the United States and GDPR for Europe). As referenced above, external electronic health record data received by the system can be stored in the personal health record of the monitored user. Further to this, the personal health record can be synchronized with one or more external electronic health records. In this way, when a relevant change is made to the personal health record, the change can (e.g., via the Allscripts API or the Veterans Affairs Health API) be provided to a corresponding external electronic health record for writing thereto. In various embodiments, as an alternative to or in addition to such synchronization, a care team member can make a given change both in the personal health record and one or more external electronic health records. The terms “electronic health record” (e.g., referring to an external electronic health record) and “personal health record” are used at various locations hereinthroughout. However, it is noted that various functionality discussed in terms of an electronic health record can be used in conjunction with the personal health record. Likewise, various functionality discussed in terms of the personal health record can be used in conjunction with an electronic health record. As one example, the data drawn from electronic health records discussed in terms of machine learning operations can be data drawn from the personal health record. As just another example, the discussed scrubbing described in terms of the personal health record can be performed in conjunction with an electronic health record.

In various embodiments, the personal health record can be made available to care team members in a visited location (e.g., a foreign country). Such care team members can, as just an example, be granted temporary and/or limited scope access to the personal health record. For instance, such care team members can be granted access that is active only while the monitored user is visiting the location. As such, the system can support a monitored user who is traveling, or who moves to a new location (e.g., moves to a new country). More generally, in various embodiments the personal health record can be made available to care team members on a temporary and/or limited scope basis under various circumstances. As just an illustration, suppose a circumstance in which the monitored user has suffered an emergency at a shopping location. Here, the personal health record can be made available to care team members (e.g., Emergency Medical Technicians (EMTs) or paramedics) assisting the monitored user at the shopping location. Continuing with the illustration, the personal health record access granted to the assisting care team members can be limited in scope, for instance being limited to medical conditions, medications, and allergies listed by the personal health record. It is noted that granting care team members temporary and/or limited scope access can, in various embodiments, involve the system establishing the care team members as temporary and/or limited scope users of the system.

Turning to FIG. 3B, one or more MLMs of the system 313 (labeled “Learning” in FIG. 3B) can generate various outputs, including care recommendations 315. The care recommendations, and/or other outputs generated by the MLMs, can be stored in the personal health record 317, provided to the monitored user 319 (labeled “Patient” in FIG. 3B), and shared with individuals 321 (labeled “Consent Authorized Parties” in FIG. 3B) for whom the monitored user has consented sharing access.

Shown in FIG. 3C is an example element of the personal health record of the monitored user. As depicted by FIG. 3C, the element contains several fields 323-329. In particular, for the example of FIG. 3C input field 323 indicates that the element was spawned as a result of a conversation between the monitored user and a virtual assistant capability (e.g., where the capability poses the question “How are you feeling today?” and the monitored user speaks a reply). Then, field 325 indicates the date that the element was added to the personal health record (or the date that the conversation took place). Field 327, listing “Dementia” according to the example of FIG. 3C, indicates a preexisting symptom/condition of the monitored user according to electronic health record data taken as input by a first MLM of the system. Field 329, listing “Headache” according to the example of FIG. 3C, indicates a current symptom of the monitored user according to verbal input data taken as input by the first MLM. Then, field 331 indicates “Decline in cognitive ability” as a predicted condition/health status output of the first MLM (labeled “Learning” in FIG. 3C). This output can act as input to a second MLM which generates care recommendations. As such, field 333 indicates “Brain health games” as a care recommendation output of the second MLM.

In various embodiments, in implementing the data acquisition and linking operations, patient hub functionality and care team member hub functionality can be established. The patient hub functionality can provide a hub for incoming data regarding the monitored user, other than data of this sort which is generated by care team members. Then, the care team member hub functionality can provide a hub for data generated by care team members (e.g., data generated by care team members in connection with caring for the monitored user). Each hub can make its data available to various functionality of the system described herein, such as the alert/notification/reminder and machine learning functionality. For example, by making the data available to the alert/notification/reminder functionality of the system, such data can be analyzed for changes in the monitored user's health or care management that can warrant new or changed alerts/notifications/reminders. It is noted that, in various embodiments, some or all data utilized by the system (e.g., sensitive data) can be encrypted both when at rest (e.g., when stored by the system) and when in transit (e.g., when being transmitted by the system. In this way, benefits such as achieving System and Organization Controls 2 (SOC2) certification can accrue.

### Machine Learning Operations

As noted, the system can perform operations including generating care recommendations, and/or predicted conditions/health statuses. As also noted, the system can have access to one or more MLMs. The MLMs can be used by the system in generating the care recommendations, and/or predicted conditions/health statuses. The MLMs can also be used in performing other operations (e.g., in in determining effective approaches for providing alerts/notifications/reminders, as discussed hereinabove). In various embodiments, the MLMs can be provided by the machine learning module 129, and the care recommendations and/or predicted conditions/health statuses can be provided via the care recommendations module 109, using the machine learning module 129.

Among the MLMs provided by the machine learning module 129 can be one or MLMs which each receive inputs, and generate therefrom output indicating a predicted condition/health status of the monitored user. In some embodiments, the generated condition/health status prediction can include an urgency indicator (e.g., indicating emergency or non-urgent). Such an MLM can alternately or additionally generate as output care recommendations. As an example, such an MLM can take as input data regarding verbal input provided to the system by the monitored user. As another example, such an MLM can take as input data regarding IoT/health-monitoring device outputs. As a further example, such an MLM can take as input data drawn from electronic health records of the monitored user As an additional example, such an MLM can take as input data drawn from metadata. As just some examples, such metadata can include firmware versions (e.g., sensor firmware versions) and software versions (e.g., electronic health record software version or API versions). Such use of metadata can allow a greater totality of conditions relating to data collection to be provided to the MLM. In this way, benefits including an increased confidence level in MLM predictions can be realized. Where metadata is used as a source of MLM inputs, changes in metadata (e.g., where a sensor receives a software) can, in various embodiments, invalidate prior results or warrant reassessment. Such inputs and outputs can be in the form of tuples or vectors.

In various embodiments, the data regarding verbal input provided to the system can be keywords drawn from the verbal inputs (e.g., keywords drawn from speech provided to the virtual assistant capability, or keywords drawn from speech communications between the monitored user and care team members or family members). For instance, verbal input can be provided by the monitored user to the virtual assistant capability. A textual representation of the verbal input provided via the virtual assistant capability can be received by the machine learning module 129 (or another module) via the human interface module 131. Subsequently the machine learning module 129 can extract keywords from a text representation of the verbal input by providing it to an MLM of the machine learning module 129 which has been trained to extract from an input sentence medically relevant keywords (e.g., relevant medical terms). As just some examples, an MLM which analyzes syntax and/or semantics, and/or an RNN-based MLM can be used. As a further example, keyword extraction can be performed using the Amazon Comprehend Medical webservice. As just some examples, the extracted keywords can regard one or more of: symptoms; conditions; sociability; pain; heart rate; blood pressure; insulin/blood sugar level; sentiment; calories burnt; weight; age; height; medications; sleep; mobility; falls, neural activity; fine motor skills; and dexterity. Also, in various embodiments such keyword extraction functionality can include identifying conversational terms relating to medical conditions and the like, and providing as the extracted keywords clinically-viable terms which correspond to those conversional terms.

In various embodiments, the system can provide extracted keywords to one or more MLMs of the machine learning module 129. The MLMs can use the keywords to generate care recommendations, and/or predicted conditions/health statuses. In this way, the MLMs can generate various outputs which are relevant to the health of the monitored user. These outputs can help inform, as just some examples, what level and type of care they need, whether their current care plan adequately addresses their needs, what kind of support they need in managing care, if they require a change in medication, if they require specific services or device-based monitoring, and/or generation (e.g., MLM-based generation) of care recommendations that can improve their quality of life and aid in prevention of decline.

While extraction of keywords from a text representation of verbal input is discussed, other possibilities exist. As examples, keyword extractions can alternately or additionally be performed with respect to data generated by IoT/health-monitoring devices and/or data drawn from electronic health records. For example, the data acquisition/link module 103 can receive such electronic health records from hospitals, clinics, healthcare systems, and other sources (e.g., using the Allscripts API or the Veterans Affairs Health API). Subsequently, keywords can be extracted from the healthcare records, and the resultant keywords can be provided to one or more MLMs of the machine learning module 129. As to providing, to an MLM which generates predicted condition/health status and/or care recommendation outputs, input data regarding verbal input, IoT/health-monitoring devices, and/or electronic health care records, the following is noted. One or more of such inputs can be provided in the form of extracted keywords or not in the form of extracted keywords (e.g., in raw form), depending on the embodiment.

The MLM-generated condition/health status predictions and/or care recommendations can be written to the personal health record of the monitored user, using the storage module 127. Alternately or additionally, the MLM-generated condition/health status predictions and/or care recommendations can be written to one or more external electronic health records of the monitored user (e.g., where desired and permitted by the monitored user). Also, the condition/health status predictions and/or care recommendations can be communicated to the monitored user, to family members, and/or to care team members, via the mobile app or the virtual assistant capability. Further still, in various embodiments previously generated condition/health status predictions and/or care recommendations (and/or the personal health record of the monitored user) can be used as inputs to the MLM when generating new condition/health status predictions and/or care recommendations.

The MLMs used by the machine learning module 129 in generating condition/health status predictions and/or care recommendations can be neural network-based MLMs, such as MLP-based classifiers. Such an MLM can be trained using training sets made up of inputs and corresponding outputs. For a given element of the training set, given values of various inputs of the sort discussed can be listed. As such, a given element of the training set can list as inputs given values of verbal input-based data, IoT/health-monitoring device-based data, and/or electronic health record-based data. In some embodiments the given element of the training set can list as outputs a condition/health status and/or care recommendation considered to appropriately correspond to the inputs. For instance, the particular training set outputs listed for given training set inputs can be selected by a physician or be chosen based on an authoritative medical source, as just some examples. As further examples, third-party databases, symptom checker data, academic content, and/or population health management data can be used in generating training data sets. In this way, the MLM can, once trained according to the training set, be able to output condition/health status predictions and/or care recommendations when presented with a set of inputs. Also, in various embodiments the MLM can be further trained subsequent to deployment. In such embodiments, where a care team member receives a condition/health status prediction and/or care recommendation generated by the MLM, the care team member can be invited by the app or virtual assistant capability to indicate whether they agree with the MLM's output. Where they do not, the app or virtual assistant capability can invite them to provide an alternative condition/health status prediction and/or care recommendation. The results of this interaction with the care team member can be used in generating further training sets for the MLM. Although the alternative condition/health status predictions and/or care recommendation has been discussed as being provided by a care team member, other possibilities exist. For example, in various embodiments the alternative condition/health status predictions and/or care recommendation can alternately or additionally be provided by the monitored user, or by a family member. In this way, advantages such as improving the precision and recall of the MLM over time can accrue. It is noted that, hereinthroughout, training of MLMs can utilize relevant and/or high-quality training data.

Discussed has been an example where an MLP-based classifier receives as input one or more of: a) data regarding verbal inputs to the system; b) data generated by IoT/health-monitoring devices; and c) data regarding electronic health records. Further discussed has been this classifier generating as output a predicted condition/health status, and/or a care recommendation. However, other possibilities for inputs and outputs to the classifier exist. For instance, such an MLP-based classifier can act to receive as input one or more of the noted three elements, along with also a given predicted condition/health status, or a care recommendation. This classifier can then generate as output an indication of whether or not the inputted condition/health status/care recommendation is predicted to apply to the monitored user, given the other three one or more inputs.

Such a classifier can be trained according to a training set whose elements list as inputs given values for the noted inputs, and that list as output indication of whether or not the inputted condition/health status/care recommendation is considered to apply, given the other inputs. Such indication can be specified by a physician or drawn from an authoritative medical source, as just some examples. Also, in various embodiments such a classifier can be further trained subsequent to deployment. In such embodiments, where a care team member receives a condition/health status predictions and/or care recommendation generated by the classifier, the care team member can be invited by the app or virtual assistant capability to indicate whether they agree with the output of the classifier. For example, the care team member can reply by providing a thumbs-up or a thumbs-down via tap or voice. The results of this interaction with the care team member can be used in generating further training sets for the classifier. In particular, a training set element—which lists as inputs the inputs which led to the classifier output, and which lists as output an indication of the thumbs-up or thumbs-down—can be added. Although the thumbs-up/thumbs-down has been discussed as being provided by a care team member, other possibilities exist. For example, in various embodiments the thumbs-up/thumbs-down can alternately or additionally be provided by the monitored user, or by a family member.

Then, although the use of an MLP-based classifier has been discussed, other types of MLMs can be used in generating the condition/health status predictions and/or care recommendations. For example, decision tree classifiers can be used. Also, in various embodiments unsupervised clustering can be used in the generation. Further, although utilizing one or more MLMs of the machine learning module 129 is discussed, other possibilities of generating the condition/health status predictions and/or care recommendations exist. For example, in various embodiments one or more web services and/or external data sources can be used in the generation. Although various types of data have been discussed as being used as MLM inputs, such data types are merely examples, and other types of data can be used. For example, data acquired by the registration/billing/settings module 123 can, in various embodiments, be used as a data source for MLM inputs. Further, it is noted that in various embodiments the machine learning approaches discussed hereinthroughout can utilize correlations between multiple inputs to achieve benefits including but not limited to reducing false negatives, reducing false positives, and discovering new multifactorial predictors. In this way, the use of multiple inputs by the system can achieve improved results versus, for instance, using separate inputs (e.g., separate sensor inputs).

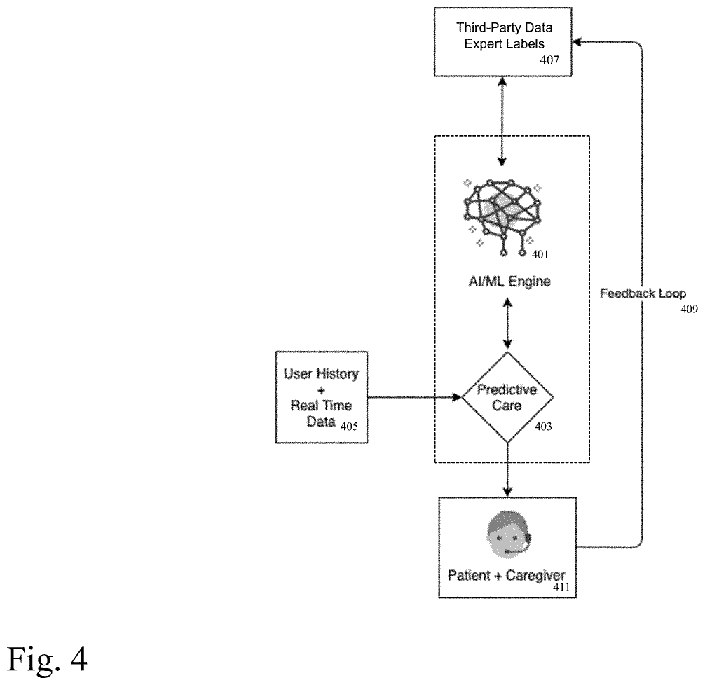

FIG. 4 shows an example of machine learning functionality. As depicted by FIG. 4, the system can utilize one or more MLMs 401 (labeled “AI/ML Engine” in FIG. 4) in generating the care recommendations 403 (labeled “Predictive care” in FIG. 4). As also depicted by FIG. 4, inputs used by the one or more MLMs in generating the care recommendations can include (405) data drawn from electronic health records and data generated by IoT/health-monitoring devices (labeled “User History+Real Time Data” in FIG. 4). Then, as depicted by FIG. 4, the one or more MLMs can be trained using training sets that include (407) as outputs care recommendations specified by physicians or drawn from authoritative medical sources (labeled “Third-Party Data Expert Labels” in FIG. 4). Further still, as depicted by FIG. 4 the one or more MLMs can be further trained subsequent to deployment, such as via feedback 409 provided by monitored users and care team members 411 in response to care recommendations (labeled “Feedback Loop” and “Patient+Caregiver” in FIG. 4).

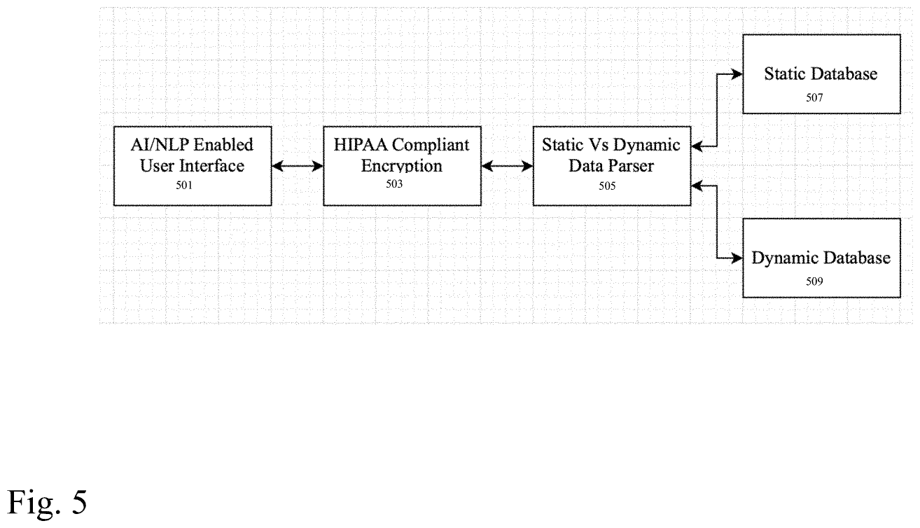

As noted, MLM inputs can include data regarding verbal input provided to the system. Turning to FIG. 5, at step 501 machine learning (labeled “AI” in FIG. 5) and/or natural language processing (NLP) can be used to convert the verbal inputs to text. As one example, where an Amazon Alexa skill is used to receive the verbal inputs, the skill can make available a textual representation of the verbal inputs (e.g., with the textual representation being passed to the human interface module 131). As another example, a web service such as Amazon Transcribe or Amazon Transcribe Medical can be used to generate a textual representation of the verbal inputs. As yet another example, one or more MLMs of the system can be used to generate a textual representation of the verbal inputs. Also at step 501, keywords can be drawn from the verbal inputs. Further at step 501, electronic health record data and data generated by IoT/health-monitoring devices can be acquired. At step 503, encryption of the intaken data can be performed. As just one example, the encryption can be HIPAA-compliant. Such encryption can be performed by the storage module 127. At step 505, the storage module 127 can parse the intaken data into static data and dynamic data. As examples, the static can include name, age, and historical conditions of the monitored user. The dynamic data can, as examples, include current symptoms and IoT/health-monitoring device data. Then, at steps 507 and 509 the storage module 127 can perform segregated data archiving/storage, such that the parsed static data is archived/stored separately from the parsed dynamic data (e.g., the static and dynamic data can be stored in different databases). It is noted that, in various embodiments, the parsing and the segregated archiving/storage does not occur (e.g., the noted data can be stored together). Subsequently, the data of the static database 507 and the dynamic database 509 can be used as inputs to one or more MLMs of the system.

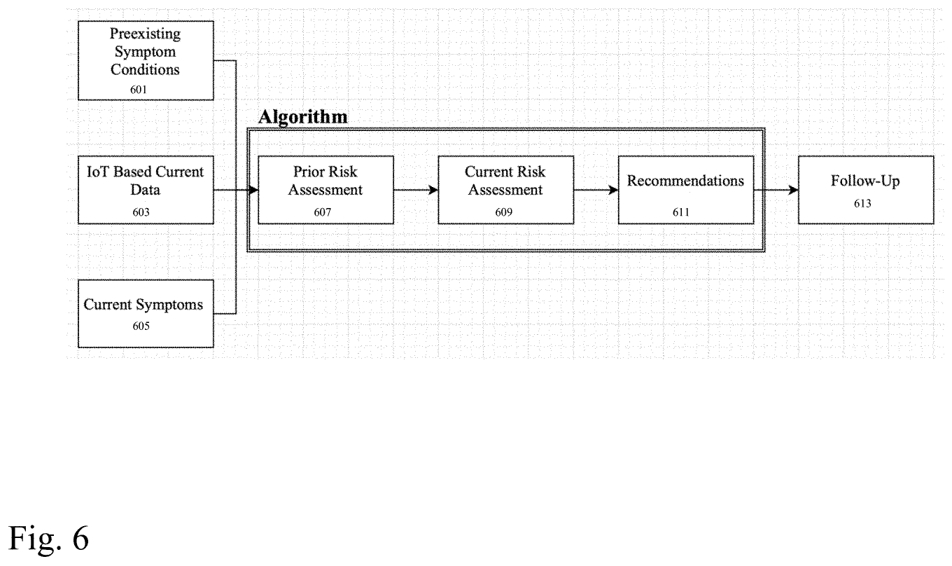

Turning to FIG. 6, an MLP-based classifier of the sort noted, which can output care recommendations, is discussed. As depicted by FIG. 6, the classifier can receive as input data regarding electronic health records 601 (labeled “Preexisting Symptom Conditions” in FIG. 6). This input can be derived from historical medical records, and can regard, as just an example, medications prescribed. In some embodiments, such data can be obtained from static storage. The classifier can also receive as input data generated by IoT/health-monitoring devices 603 (labeled “IoT Based Current Data” in FIG. 6). This input can include timestamped data and location data (e.g., GPS and Bluetooth beacon data). The data can be obtained regularly from a smartphone of the monitored user and/or sensors worn by the monitored user. In some embodiments, the data can be obtained from dynamic storage. Further, the classifier can receive as input data regarding verbal inputs to the system (or inputs provided via the mobile app) 605 (labeled “Current Symptoms” in FIG. 6). This input can include manually recorded current symptoms, and/or qualitative observations (e.g., fever and cough). In some embodiments, the data can be obtained from dynamic storage.

Using the inputs, the classifier can generate (607, 609) as output a care recommendation 611. The care recommendation can be presented to the monitored user, care team members, and/or family members via the virtual assistant capability and/or via the app. An example of a presented care recommendation can be that the monitored user follow up with a medical professional regarding their health (613).

In FIG. 6, the label “Prior Risk Assessment” indicates the classifier making use of the data regarding electronic health records and the IoT/health-monitoring device data (e.g., less-recent IoT/health-monitoring device data) as inputs when generating the care recommendation. Then, the label “Current Risk Assessment” in FIG. 6 indicates the classifier making use of the data regarding verbal inputs to the system (or inputs provided via the mobile app) and the IoT/health-monitoring device data (e.g., more-recent IoT/health-monitoring device data) as inputs when generating the care recommendation. Although the use of an MLP-based classifier is discussed here, other possibilities exist. For instance, in general, data analysis entailing one or more algorithms that use machine learning or other analytical methods to assess risk from historical and/or current symptoms can be used. Also, in various embodiments the care recommendation generated by the classifier can be cross referenced with insights gathered by a third-party algorithm (e.g., accessed as a web service), such as one that uses machine learning or other analytical methods.

As noted, the MLP-based classifier can utilize verbal inputs in generating the care recommendation and predicted condition/health status outputs. As an example, the verbal inputs can be provided by the monitored user via a virtual assistant capability. As just an example, the capability can pose to the monitored user a question such as “how are you feeling today?.” As just an illustration, where the virtual assistant capability is an Amazon Alexa skill, words that the monitored user utters in describing how they feel can correspond to an Alexa slot. Further, the virtual assistant capability can be configured to have a speech-to-text conversion result of the utterance passed to the human interface module 131 (e.g., via an HTTP API thereof). As another illustration, the virtual assistant capability can provide a recording of the utterance to the system, and the system can subject the recording to speech-to-text conversion. Subsequently, the machine learning module 129 can receive the speech-to-text result, and utilize it in providing the input data regarding verbal input to the classifier. As another example, the verbal inputs can be drawn from communications (e.g., calls) between the monitored user and family members or care team members. The system can apply a speech-to-text conversion to the audio component of such communications so as to yield a corresponding transcript. Subsequently, the machine learning module 129 can receive the speech-to-text result, and utilize it in providing the input data regarding verbal input to the classifier.

With further regard to the MLP-based classifier utilizing verbal inputs in generating the care recommendation and predicted condition/health status outputs, the following is noted. As just an example, such functionality can include the system intaking audio conversations, in which the patient is speaking, through a voice interface from a mobile device or an IoT device (e.g., an Amazon Echo). For instance, the conversation can be the device asking the question “how are you feeling today?,” and the monitored user speaking a reply. The conversation can be transcribed to text. As one example, the transcription can occur in real-time (e.g., where an Alexa skill is used as discussed above). As another example, the conversation can be transcribed subsequent to a recording thereof. For example, a recording of the conversation can be received from the device at the human interface module 131. The human interface module 131 (or another module) can then obtain a transcription of the recording (e.g., via AWS Transcribe Medical). Subsequently, the transcribed text can be analyzed for indicators of health such as keywords relating to health symptoms, conditions, sentiment, nutrition, fitness, health data, social indicators, and indicators of cognitive ability. Such keyword extraction can be performed as discussed above (e.g., using the noted RNN-based MLM, or using Amazon Comprehend Medical). Then, the generated keywords can be provided to the MLP-based classifier. The classifier can subsequently use the keywords in generating care recommendation and predicted condition/health status outputs.

In this way, the system can analyze conversations with monitored users, using natural language processing to extract keywords that are indicators of health. By providing these keywords to the classifier and receiving the noted outputs therefrom, the system can help prevent decline through value-based care. The use of conversational artificial intelligence (AI) via the virtual assistant capability can extend an interface with increased accessibility for impaired users, such as but not limited to, those who are visually impaired. An example of such a communication is the monitored user sharing their current symptoms via voice with the virtual assistant capability (e.g., in response to the capability posing the question “How are you feeling today?”). The reply of the monitored user can be handled in the manner discussed, so as to allow the reply to be used in connection with an input to the MLP-based classifier. Alternately or additionally, in some embodiments the reply of the monitored user can be used in connection with a third-party symptom checker webservice or database, in order to receive care recommendations and predicted condition/health status outputs therefrom.

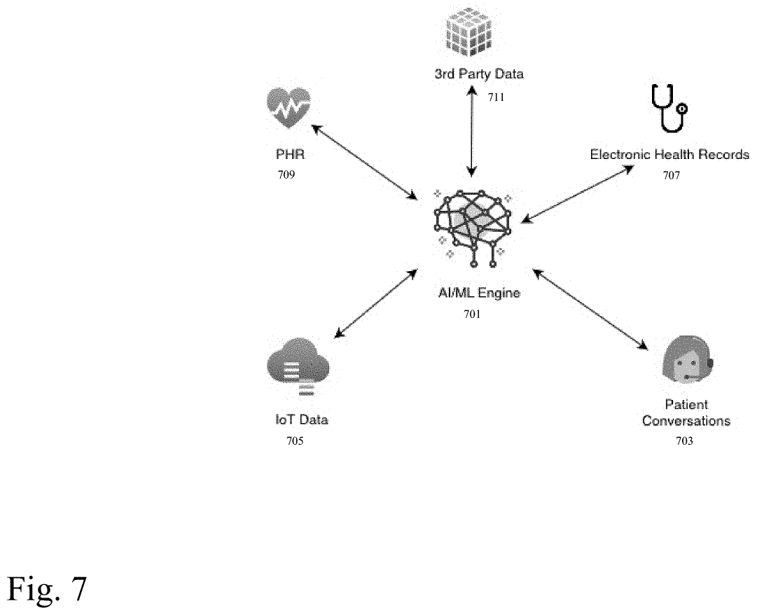

As referenced above, the MLP-based classifier can use various inputs to generate care recommendation and predicted condition/health status outputs. Turning to FIG. 7, the MLP-based classifier 701 (labeled “AI/ML Engine” in FIG. 7) can receive various inputs in generating the noted outputs. In particular, the inputs can include: a) data regarding verbal input 703 (labeled “patent conversations” in FIG. 7); b) data generated by IoT/health-monitoring devices 705 (labeled “IoT Data” in FIG. 7); c) data drawn from electronic health records 707; d) data relating to the personal health record of the monitored user 709 (labeled “PHR” in FIG. 7); and e) third-party data 711. As just one example, the third-party data can correspond to the result of the system providing various information known about the monitored user to a third-party symptom checker webservice, receiving information about the monitored user therefrom, and utilizing the received information to provide input to the classifier. In this way, use of the third-party data can allow for richer inputs to be provided to the classifier, and for potential benefits such as enhanced classifier performance to accrue.

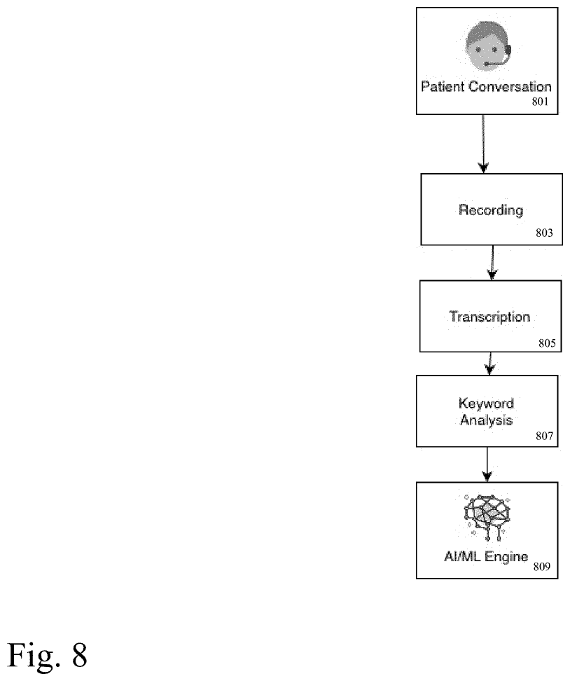

Turning to the example of FIG. 8, at step 801 a conversation between the monitored user and the virtual assistant capability (or mobile app) can occur. For instance, the conversation can be the virtual assistant capability/app asking the question “how are you feeling today?,” and the monitored user speaking a reply. Then, at step 803, the system can receive from the virtual assistant capability or from the mobile app a recording of the monitored user's reply. At step 805 the system can obtain a transcription of the recording (e.g., via AWS Transcribe Medical). Further, at step 807 the system can perform keyword extraction with respect to the transcription. Then, at step 809 the system can provide the keywords to the MLP-based classifier (labeled “AI/ML Engine” in FIG. 8). Subsequently, the MLP-based classifier can use the keywords in generating care recommendation and predicted condition/health status outputs of the sort noted.

As just an illustration, a predicted condition/health status can be that the monitored user appears to be afflicted with a decline in neural activity. On the other hand, continuing with the illustration, a care recommendation can be that the monitored user partake in brain games. As discussed above an MLM can directly generate care recommendations from verbal-based, IoT/health-monitoring device-based, and electronic health record-based inputs. However, other possibilities exist. For example, a first MLM (e.g., an MLP-based classifier) can generate as output predicted conditions/health statuses, from verbal-based, IoT/health-monitoring device-based, and electronic health record-based inputs. Then, a second MLM can receive, as its input, the output of the first MLM. The second MLM can use this input to generate a care recommendation. As on example, the second MLM can be an MLP-based classifier. As another example, the second MLM can be a decision tree-based model. The generated care recommendations can be shared with the monitored user, care team members, and family members (e.g., with the consent of the monitored user and in a HIPAA-compliant fashion).

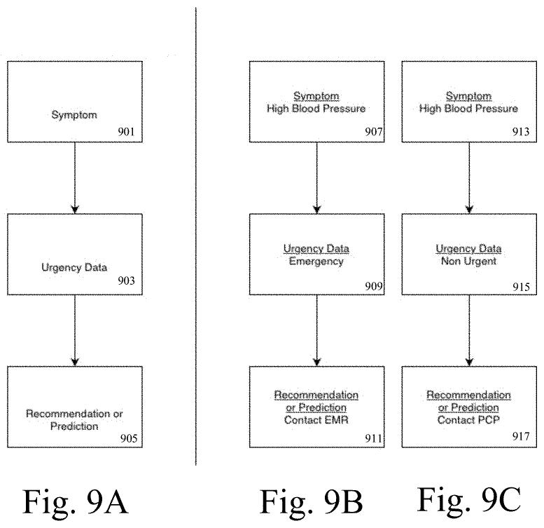

Turning to the example of FIG. 9A, a first MLM can generate a predicted condition/health status 901 (labeled “symptom” in FIG. 9A). The generated condition/health status prediction can include an urgency indicator 903 (labeled “Urgency Data” in FIG. 9A). A second MLM can take the output of the first MLM as input and generate therefrom a care recommendation output 905 (labeled “Recommendation or Prediction” in FIG. 9A).

In FIGS. 9B and 9C, specific examples of the functionality of FIG. 9A are set forth. In FIG. 9B, the first MLM generates “high blood pressure” as its predicted condition/health status 907. The first MLM also outputs “emergency” as its urgency indicator 909. Then, the second MLM takes the output of the first MLM as input and generates therefrom “contact emergency medical response (EMR)” as its care recommendation 911. Then, in FIG. 9C, the first MLM generates “high blood pressure” as its predicted condition/health status 913. The first MLM also outputs “non-urgent” as its urgency indicator 915. Then, the second MLM takes the output of the first MLM as input and generates therefrom “contact primary care provider (PCP)” as its care recommendation 917.

The system can utilize one or more MLMs (e.g., RNN-based MLMs) to perform language translation. Alternately or additionally, the system can utilize a web service such as Amazon Translate to perform such translations. As just an example, the system can be internally focused on the English language, but utilize translation to receive inputs from and provide inputs to non-English speakers. As another example, the system can translate non-English languages into English for clinical viability, such as when writing to personal health record for the monitored user. The system can also intake, store, and output through other languages without translating to English. For example, the human interface module 131 can provide language and localization functionality in this regard. Languages between which the system can provide translations can include English, Hindi, Japanese, Chinese, Spanish, and French, as just some examples. Also, in various embodiments translation can include converting conversation terms regarding medical conditions and the like to clinically viable terms. As just an example, a decision tree MLM can be used for this purpose. Also, in various embodiments the system can provide real-time bidirectional translation functionality. Such functionality can be employed, as just one example, to allow communication among individuals (e.g., care team members) who speak different languages.

### Care Coordination Operations

The system can perform operations including aiding in the coordination of care for the monitored user. As just some examples, the system can provide calendar functionality, messaging portal functionality, calling functionality, care directory functionality, and communication log functionality. In various embodiments, the system can provide such functionality via the care coordination module 111.

Turning to the calendar functionality, as noted above the system can provide various time-based alerts, notifications, and reminders, such as a reminder that a medication dosing is coming due. The calendar functionality can provide for the viewing and setting of such alerts/notifications/reminders, for instance via the virtual assistant capability or via the mobile app. The system can allow the alerts/notifications/reminders to be viewed and set by the monitored user. Also, with the consent of the monitored user the alerts/notifications/reminders can also be set by family members and by care team members.

As just some examples, alerts/notifications/reminders supported by the system can regard: a) medication dosings; b) medication deliveries; c) transportation pick-ups/drop-offs; d) exercise; e) nutrition; f) health/fitness/wellness games (e.g., brain health games); g) telehealth visits; h) care team member (e.g., professional caregiver) visits; i) calls (e.g., with care team members or family members); and j) general alerts/notifications/reminders. In various embodiments, the system can provide a notification or reminder when an event is upcoming, and an alert if the event is missed. Alerts/notifications/reminders can be provided to the monitored user, and can with the consent of the monitored user be shared with family members and care team members. The discussed alert/notification/reminder functionality can be implemented in a fashion compliant with local regulations regarding health data privacy (e.g., HIPAA). In some embodiments, the alert/notification/reminder functionality can utilize the Google Calendar API.

The messaging portal functionality can provide text, audio chat, and/or video chat capabilities which allow the monitored user, family members, and care team members (e.g., clinicians and pharmacies) to communicate with one another. As an example, the text/audio/video chat functionality provided by the system can make use of WebRTC (Web Real-Time Communication).

The system can allow for both group chats and individual chats. By way of these chats, benefits including allowing care of the monitored user to be managed more effectively can accrue. In various embodiments, the system can record the chats with the permissions of the monitored user and other parties (e.g., where required by law). Also, messaging can, in various embodiments, be tied to calendar data (e.g., in the form of appointments), or health data, such as symptoms.

Further to the messaging portal functionality, the system can provide the noted calling functionality. The calling functionality can integrate the system with calling functionality built into a device, such as Apple Facetime or cellular telephone call functionality. The mobile app can offer an in-app and/or click-to-call feature which allows for integration with built-in device calling functionality. Likewise, the virtual assistant capability can offer voice commands which allow for integration with built-in device calling functionality (e.g., the virtual assistant capability and the mobile app can work in conjunction to allow built-in call capabilities of the device upon which the app runs to be accessed by voice via the virtual assistant capability). To facilitate discussion, certain functionality is discussed in connection with calls while other functionality is discussed in connection with text, audio, and/or video chat. However it is noted that, in various embodiments, functionality discussed in connection with calls can instead be implemented in connection with text, audio, and/or video chat. Likewise, in various embodiments functionality discussed in connection with text, audio, and/or video chat can instead be implemented in connection with calls.

With further regard to the calling functionality, the system can automatically place calls on behalf of the monitored user. The circumstances under which the system automatically places calls can include emergent and non-emergent situations. As just one illustration of an emergent-circumstance automatic call, the system can, as discussed, have the capability of recognizing that the monitored user has fallen. Continuing with the illustration, under this circumstance, the system can initiate a call to emergency services (e.g., dialing 911). Further examples of emergent circumstances under which the system can place a call include cardiac arrest, stroke, loss of consciousness, and an asthma attack. Here, cardiac arrest can be detected in a fashion including, for example, receiving an electrocardiogram (ECG) signal from a watch worn by the monitored user (e.g., via Apple HealthKit), and passing the signal to an MLM of the system capable of taking an ECG as input and outputting a predicted potential cardiac diagnosis. Then, stroke can be detected in a fashion including receiving speech spoken by the monitored user (e.g., speech provided to the mobile app or the virtual assistant capability), and passing a recording of the speech to an MLM of the system capable taking an audio recording data as input and outputting a prediction of whether or not the speech is slurred. Loss of consciousness can be detected in a fashion including the system recognizing a lack of response from the monitored user to a query spoken by the virtual assistant capability (e.g., the capability can periodically post the query “Please confirm that you are ok.”). Further, asthma attack can be detected in a fashion including collecting ambient audio (e.g., via a microphone associated with the mobile app or the virtual assistant capability), and passing a recording of the ambient audio to an MLM of the system capable of taking audio recording data as input and outputting a prediction of whether or not the audio depicts asthmatic breathing. In various embodiments, using the virtual assistant capability to receive an audio recording can involve employing a custom mobile app which acts as front-end to the virtual assistant capability. Alternately or additionally, in various embodiments using the virtual assistant capability to receive an audio recording can involve having the virtual assistant capability connect to the system via a telephone call, Skype call, audio chat, or the like.

As just one illustration of a non-emergent-circumstance automatic call, the system can use the noted calendar functionality to recognize that the monitored user has an upcoming well-patient (or other) telehealth visit. Continuing with the illustration, under this circumstance the system can initiate a call to the care team member providing the telehealth visit. Such a care team member can be a member of the system, and have their system account linked to the account of the monitored user. Where an automatic call regards a non-emergent situation, the system can secure permission (e.g., via the virtual assistant capability) from the monitored user before calling. Where an automatic call regards an emergency situation, the system can place the call without securing permission. In various embodiments, such a call can be directed towards an emergency telephone number (e.g., 911 in the US or 999 in the UK). Also, in various embodiments, the system can use text-to-speech capability speak the location of the monitored user to the called party. The system can, as just one example, utilize GPS capability of a smartphone or IoT device of the monitored user in determining the location.

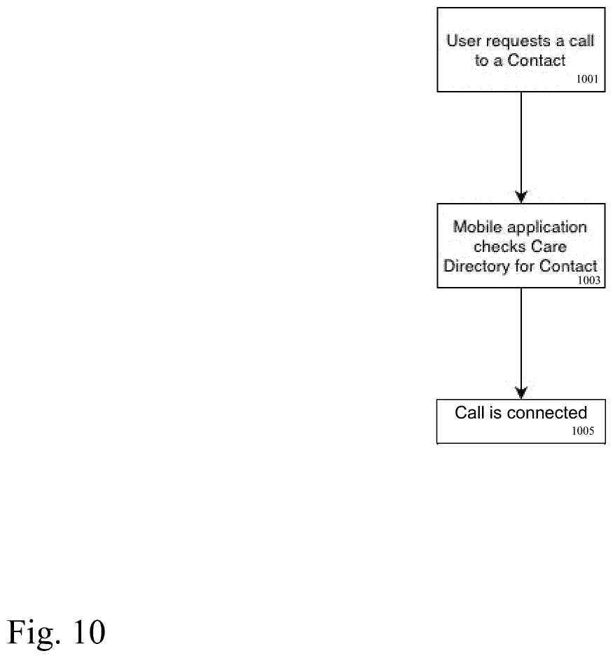

Further, beyond automatic calls, the system can allow for voluntary calls where the monitored user requests (e.g., via the virtual assistant capability or the app) that a call be made. Such calls can include calls to individuals listed in the below-discussed care directory. Turning to FIG. 10, at step 1001 the monitored user can request that a call be made to an individual listed in the care directory. As just an illustration, the monitored user can speak to the virtual assistant capability “Call Dr. Bill,” where “Dr. Bill” corresponds to an entry in the care directory. Then, at step 1003 the system can search the care directory for the relevant contact. Continuing with the illustration, the care directory can associate the text “Dr. Bill” with a given telephone number. Subsequently, at step 1005 the system can utilize built-in calling functionality of a device of the monitored user to connect the call. In various embodiments, the system can follow an escalation procedure where the call fails to connect (e.g., fails to connect after a predetermined number of tries, such as one try). In these embodiments, where the call fails to connect, the system can send a notification to care team members and family members. Also in these embodiments, where the call fails to connect and the call regards an emergency, the system can send an alert to the monitored user and to linked individuals designated by the monitored user (e.g., designated via monitored user/patient settings). It is noted that the communication capabilities of the system can, as just one example, allow a traveling family member or care team member to stay in contact with a monitored user who stays behind in a home country. As just another example, the communication capabilities of the system can allow a traveling monitored user to be put into contact with family members or care team members back home, or to be put into contact with care team members of a visited location (e.g., a foreign country).

In various embodiments, the system can perform triage operations to put the monitored user in contract with an appropriate care team member. As just one example, as referenced above the system can extract medically related keywords from verbal inputs provided to the system. Such medically related keywords can be provided to an MLM (e.g., an MLP-based classifier) of the machine learning module which has been trained to take medically related keywords as input, and output a medical professional type and/or a physician type. As an illustration, when provided with input keywords including “chest pain” or “high blood pressure,” the MLM can output an indication of a cardiologist. As another illustration, when provided with input keywords including “rash” or “itching,” the MLM can output an indication of a dermatologist. Utilizing such an output, the system can consult the care directory to determine a care team member who matches the output of the MLM (e.g., locating a cardiologist in the care directory where the MLM outputs indication of a cardiologist). Subsequently, the system can act to put the monitored user in contact with the determined care team member. As just an example, the system can use the app or the virtual assistant capability to suggest to the monitored individual that the determined care team member be called. Where the monitored user agrees, the system can connect a call to the determined care team member in the manner discussed. It is noted that, in various embodiments, the care team member that the system selects from the care directory can have a prior and/or agreed-upon care team member-patient care relationship (e.g., doctor-patient care relationship) with the monitored user. It is further noted that, in various embodiments, the system can select a group practice rather than a particular care team member. In these embodiments, the system can act to put the monitored user in contact with the group practice such that any care team member on call for the practice can respond to the call. Such a selected group practice can be one with which the monitored user has a care team member-patient care relationship. Moreover such a group practice can, as just an example, be one that has signed up with a corresponding payer.

Triage operations have been discussed in terms of providing, to the MLM, medically related keywords obtained from verbal inputs. However, it is noted that, in various embodiments other data can alternately or additionally be provided to the MLM (e.g., data regarding IoT/health-monitoring device outputs and/or data drawn from electronic health records). Also, it is noted that, in various embodiments, the triage functionality of the system can be used in conjunction with the automatic calling functionality of the system. As just an example, suppose that the system has detected that the monitored user has fallen and is going to place an automatic call on behalf of the monitored user. Continuing with the example, where the MLM has outputted indication of a cardiologist in response to keywords provided to it, the system can place the automatic call to a cardiologist listed in the care directory.

Via the care directory functionality, the system can allow the monitored user, family members, and care team members to have access to vital information, with those individuals being able to access such information via the mobile app and via the virtual assistant capability. In particular, the system can store (e.g., via the storage module 127) contact information for the monitored user, as well as contact information for users linked in the system to the monitored user. Users linked to the monitored user can, for instance include care team members (e.g., pharmacies and physicians) and family members. As such, the care directory, as just some examples, include physical addresses of offices, clinics, homes, or hospitals. In various embodiments, the care directory can store information regarding care team members in different areas (e.g., different countries). In this way, the system can support a monitored user who is traveling.

The contact information can, as just some examples include name, personal and/or business address, personal and/or business telephone number, personal and/or business email address, and personal and/or business messaging address. In various embodiments, where contact information includes an emergency number (e.g., 911 or 999), the system (or smartphone, IoT, or other device utilized by the system in making calls) can be registered with a corresponding telecom provider. Such registration can, as just an example, establish that a given location (e.g., the home of the monitored user) is to be the default for the location to be associated with an outgoing call to an emergency number. Also, in various embodiments a GPS-based location can be associated with an outgoing call to an emergency number (e.g., a smartphone-derived GPS location). Further, for a given user other than the monitored user, the contact information can include relationship of that user to the monitored user, notes relating to interactions between that user and the monitored user (e.g., clinical setting notes), and medicines prescribed for the monitored user by that user.

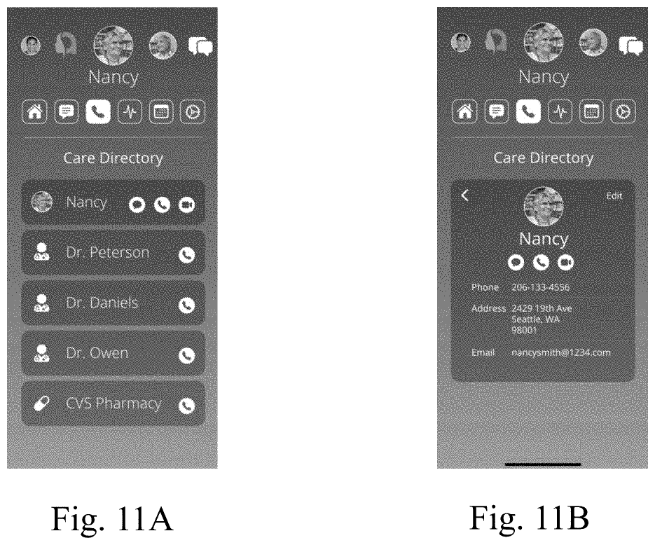

The system can allow the monitored user, as well as other users of the system, access to the care directory via the virtual assistant capability and via the app. The virtual assistant capability can provide access to the care directory by answering voice queries. The app can provide access to the care directory by allowing browsing and searching of the care directory via a UI. Shown in FIGS. 11A and 11B are two screenshots of examples of the mobile app providing access to the care directory.

Turning to the communication log functionality, the system can store (e.g., via the storage module 127) various data relating to calls and messaging between the monitored user and care team members and family members. Further, via the communication log functionality the system can store various data relating to conversations between the monitored user and the system, via the virtual assistant capability. In various embodiments, the system can store such data (e.g., data relating to calls/messaging between the monitored user and care team members) to the personal health record of the monitored user. Further, via the above-discussed synchronization between the personal health record and one or more external electronic health records, this data can be added to one or more appropriate external electronic health records. In this way, benefits such as reducing potential redundancy and duplicate work for care team members (e.g., physicians) can accrue.

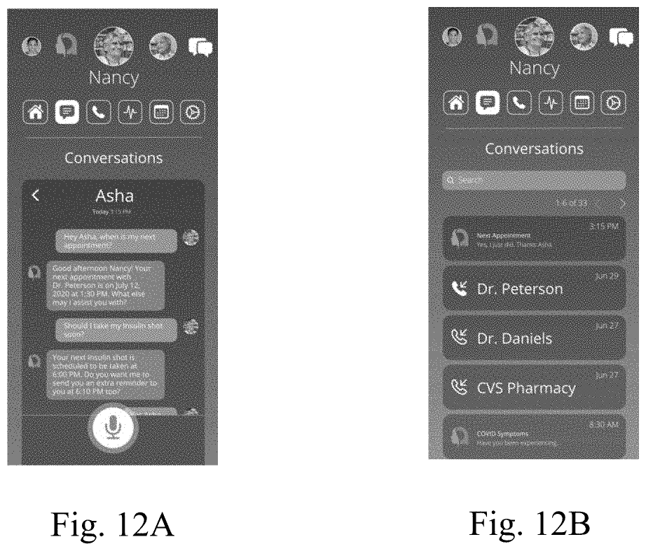

The stored information regarding the calls and messaging can include metadata such as number called, address messaged, communication date, and communication duration. As just an illustration, where the mobile app is an Android mobile app, for a cellular phone call the number called, date of call, duration of call, and name of called individual can be accessed, respectively, via the NUMBER, DATE, DURATION, and CACHED_NAME fields of the CallLog.Calls data structure. Likewise, the stored information regarding conversations with the virtual assistant capability and include metadata such as date and duration. The stored information regarding the calls, messaging, and virtual assistant conversations can also include content (e.g., with the consent of the participant parties). As such, the stored information can include corresponding text, audio, and/or video. In various embodiments, audio content can be transcribed into text (e.g., via the Amazon Transcribe web service). The stored metadata and content information can be—in agreement with corresponding consents—be accessible by the monitored user, care team members, and family members. Accordingly, for example, the communication log functionality can allow a care team member to read transcripts of calls and messaging that the monitored user has had with that care team member and with other care team members. As another example, the care team member can read transcripts of conversations that the monitored user has had with the virtual assistant capability. Shown in FIG. 12A is an example mobile app screenshot of a transcript of a conversation between the monitored user and the virtual assistant capability. Shown in FIG. 12B is an example mobile app screenshot of various metadata regarding calls, messaging, and conversations with the virtual assistant capability.

Further, such transcripts can be employed in conjunction with the machine learning capabilities of the system so as to yield care recommendations, and/or predicted conditions/health statuses. In particular, in agreement with that which is discussed hereinabove, keywords can be extracted from the transcripts. Further in agreement with that which is discussed hereinabove, the system can provide the extracted keywords to one or more MLMs of the machine learning module 129, and the MLMs can generate care recommendations, and/or predicted conditions/health statuses. Conversely, care recommendations can be further traced and expanded into a view of the transcripts from which the keywords for that particular care recommendation were extracted for analysis.

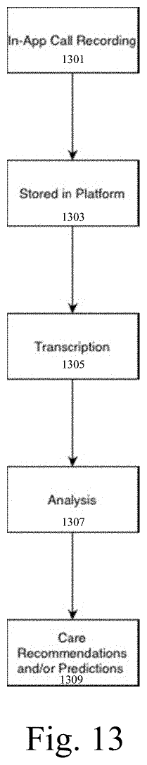

Turning to FIG. 13, at step 1301 the system can record a telephone call involving the monitored user, such as a call between the monitored user and a family member or care team member. At step 1303, the system can store the recording of the call. Then, at step 1305, the system can generate a transcript of the call. At step 1307 the system can analyze the transcript so as to generate keywords therefrom. Subsequently, the system can provide the extracted keywords to one or more MLMs of the system. At step 1309, the MLMs can generate care recommendations, and/or predicted conditions/health statuses. In various embodiments, consent can be obtained from the monitored individual ahead of recording the call and/or generating a transcript of the call. As one example, a general consent for such operations can be obtained when the monitored user registers with the system. As another example the system can (e.g., via the virtual assistant capability or the app) request such consent from the monitored individual before recording or transcribing a given call. In seeking consent, the system can remind the monitored user of the rationale for the consent (e.g., letting the monitored user know that the recording/transcribing will be used for beneficial purposes such as generating care recommendations).

### API Integration Operations

The system can perform operations including connecting with third-party services and devices (e.g., to assist in securing support services for the monitored user). In various embodiments, the system can provide such functionality via the API integrations module 113.

The system can utilize the API integration functionality to connect with third-party services and devices through an API-based integration. By connecting to these third-party services and devices, the system can import and/or export data relating to the patient's health, fitness, and/or wellness, as just some examples. In connecting with the third-party services and devices the system can use approaches compliant with local regulations (e.g., HIPAA-compliant approaches can be used).

Third-party devices with which the system connects can include IoT devices and wearables relating to health, fitness, and wellness (e.g., such a device/wearable can be a smartwatch worn by the monitored user). As examples, in connecting with such IoT devices and wearables the system can use the AWS IoT API, and/or the devices and wearables can be Alexa-enabled. In some embodiments, the system can utilize the data acquisition/link module 103 in connecting with the IoT devices and wearables.

As an example, the system can connect to a third-party service in order to pass various data collected by the system to a symptom checker webservice API, and subsequently receive diagnostic information in return. As another example, the system can pass a transcript (or other collected text) to the API of the Amazon Comprehend Medical webservice, and receive in reply extracted medically related keywords. In general, the system can connect to various third-party APIs so as to provide user experience enhancements and other specialized capabilities.

Further, various third-party support services—such as food delivery and laundry services—offer APIs, portals, and/or other communication channels. The system can access these APIs, portals, and/or other communication channels in order to assist the monitored user in ordering support services. In connecting with the support services, the system can utilize or share stored information, with the consent of the monitored user. As one example, the system can allow the monitored user to browse and order support services using the virtual assistant capability. As another example, the system can allow the monitored user to browse and order support services via the mobile app. As just some examples, support services which the system can present to the monitored user can include suggested daily activities, laundry, meal planning, nutrition, transportation, chiropractic adjustments, telehealth visits, caregiving services, handyman work, plumbing, and personal training. In various embodiments users (e.g., the monitored user, family members, and care team members) can use the virtual assistant capability or mobile app to leave reviews of used services and devices. For example, such a review can include a quantity of stars ranging between 0 and 5, along with a comment no longer than 150 characters. The system can store (e.g., via the storage module 127) such reviews in correlation with corresponding service and device vendors. The reviews can be aggregated and/or anonymized across multiple users. With further regard to telehealth visits, it is noted that such telehealth visits can include virtual physical therapy visits, virtual occupational therapy visits, and virtual physical exam visits, as just some examples. In various embodiments, data can be collected from IoT/health-monitoring devices, from device (e.g., smartphone) cameras, and/or from device (e.g., smartphone) microphones during such visits. As just an illustration, the monitored user can wear IoT sensors during virtual physical therapy or virtual occupational therapy visits. The system can capture data outputted by the IoT/health-monitoring devices, cameras, and/or microphones, and provide it to care team members (e.g., therapists or physicians) hosting the visits. In this way, the care team members can, as just some examples, perform a physical exam of the monitored user or track body movements of the monitored user. Further, the system can utilize the captured data as input to MLMs and/or as a source of data to be added to the personal health record of the monitored user, as just some examples.

In various embodiments, the system can perform various analytical or machine learning operations with regard to third-party support services. For example, the system can take into account medical problems or nutritional restrictions of the monitored user (e.g., as specified by the personal health record) when the monitored user utilizes a food delivery service. As one example, the system can use such functionality to suggest that the monitored user order only low-sodium food items where the monitored user is subject to a low-sodium dietary restriction. As another example, the system can use such functionality to alert the monitored user not to order foods that can interact with a medication that the monitored user is taking (e.g., where the monitored user is taking a CYP450 inducer or inhibitor, the system can warn the monitored user not to order grapefruit juice). Such suggestions and warnings can be provided to the monitored user via the virtual assistant capability or the app. For instance, returning to the example of the monitored user having a low-sodium dietary restriction, the system can highlight low-sodium foods in the UI of the app when the monitored user is viewing the offerings of a food delivery service.

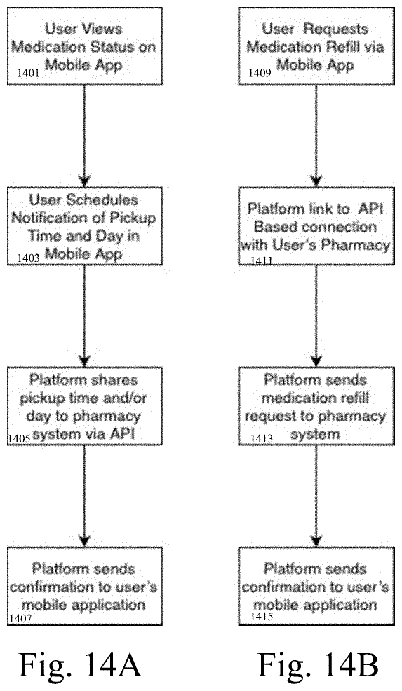

As a further example, the system can interface with the APIs (or electronic health records) of various pharmacies to perform, on behalf of the monitored user, operations including requesting medication refills, scheduling medication pick-ups, and checking to see if a prescription is ready for pick-up or has been picked up. As examples, the system can allow the monitored user to request such operations via the virtual assistant capability or via the mobile app. As just an illustration, the system and utilize the Walgreens Pharmacy Prescription API in implementing such functionality. FIGS. 14A and 14B, shown two examples of the system interfacing with a pharmacy on behalf of the monitored user. Turning to FIG. 14A, at step 1401 the monitored user (listed as “user” in FIG. 14A) can use the mobile app of the system to view an indication of whether or not a given prescription is ready for pick-up. Here, the system can have obtained such status from an API of the corresponding pharmacy. According to the example of FIG. 14A, the system can have informed the monitored user that the prescription is ready for pick-up. At step 1403, the monitored user can use the mobile app to specify a particular day/time that they desire to pick-up the prescription. The monitored user can also request that the system remind them when such pick-up day/time approaches. At step 1405, the system (listed as “platform” in FIG. 14A) can interface with the API of the pharmacy to indicate to the pharmacy the desired pick-up day/time. In response, the system can receive from the pharmacy indication that the desired pick-up day/time is granted. At step 1407, the system can use the mobile app to inform the monitored user that the desired pick-up day/time is granted. Further, the system can establish a corresponding reminder (e.g., via the alerts/notifications/reminders module 105). Turning to FIG. 14B, at step 1409 the monitored user (listed as “user” in FIG. 14B) can use the mobile app of the system to request a medication refill. At steps 1411 and 1413, the system (listed as “platform” in FIG. 14B) can interface with the API of a corresponding pharmacy to indicate to the pharmacy the desired refill. In response, the system can receive from the pharmacy an indication that the refill request has been accepted. At step 1415, the system can use the mobile app to inform the monitored user that the refill request has been placed. The system can also inform one or members of the care team that the refill request has been placed. Further, the system can provide a notification to one or more members of the care team, and/or to one or more family members, once the refill has been picked up or delivered.

In various embodiments, third-party integrations can be done via integration through platform-specific share functionality such as Share Sheets on iOS or Intents on Android. Via this share functionality, the monitored user can be directed from the mobile app of the system to an app/website of a given third-party service. Further, in various embodiments approaches other than such share functionality can be used (e.g., where integration cannot be done via such share functionality, or where it is desired to bypass such share functionality). These other approaches can involve the establishment of specific backend partnerships whereby the system books or provides a third-party service as a proxy (e.g., utilizing an API offered by the third-party service, or interfacing with a website of the third-party service via screen scraping), rather than forwarding the user to the partner app/website.

### Forums Operations

The system can perform operations including hosting various forums. These forums can include forums which allow monitored users and members of different families to discuss various issues with one another. These forums can also include forums which allow members of different care teams to discuss various issues with one another. These forums may additionally allow thought leaders to discuss topics with other users. In various embodiments, the system can provide such functionality via the forums module 115. The forums can be accessible via the mobile app, the web app, and/or via the virtual assistant capability, as just some examples.

The forum which allows monitored users and members of different families to discuss issues with one another can be termed the “community portal.” Through the community portal feature, users can gain insight into best practices in patient care and health management by browsing user-generated content and posting their own comments and/or questions. The community portal feature can be organized by condition-specific channels, and can also include a local channel that can facilitate communication among users who are located nearby one another geographically. The community portal can be implemented such that comments can be stored (e.g., via the storage module 127) as independent entities that can contain media, text, and links (e.g., links to other comments, external websites, or on-platform content, such as through deep linking). Also in the community portal, comments can be posted by a registered user (e.g., an monitored user or a family member) and responded to by creating a new comment that belongs hierarchically to the parent comment. Further still in the community portal, comments can be searched by specific keywords contained in the text or metadata associated with the keyword such as: a) the geographic location of the comment; b) the medical condition associated with the comment; c) username, id or user specific details of an author of the comment; d) date/time of the comment; and e) embedded internal or external entities referenced by the comment. Although community portal functionality has been described with reference to supporting discussion of topics such as patient care and health management, the community portal is not limited to such uses. For example, in various embodiments the community portal can allow the monitored individual to host (or participate in) recreational classes (e.g., knitting).

The forum which allows members of different care teams to discuss issues with one another can be termed the “provider portal.” The provider portal can allow care team members (e.g., physicians, clinicians, pharmacists, nurses, clinic administrators, and) caregivers to engage with monitored users and their linked care team or family members through messaging, audio, or video calling. The provider portal can also allow care team members to receive alerts and/or notifications on the health of monitored user patients. Further still, the provider portal can allow care team members to receive care recommendations, and/or predicted conditions/health statuses (e.g., as generated via the machine learning module 129). Also, the provider portal can allow care team members to search (e.g., using names and/or conditions) a patient directory for specific patients (i.e., monitored users who are users of the system). Further still, the provider portal can allow care team members to view (e.g., using approved consent protocols) patient health data of monitored users who are users of the system. What is more, the provider portal can allow care team members to edit prescribed medications for their patients (i.e., monitored users who are users of the system). Also, the provider portal can allow care team members to share (e.g., using approved consent protocols) care plan updates for a patient (i.e., a monitored user who is a user of the system) with the care team and/or family members.

Although certain functionality has been described in connection with the community portal while other functionality has been described in connection with the provider portal, such ascriptions are merely for purposes of illustration. As such, for instance, various functionality ascribed to the community portal can be implemented in connection with the provider portal. In various embodiments, either or both of the community portal and the provider portal can be implemented via Discourse, wherein the system (e.g., via the forums module 115) accesses Discourse functionality via Discourse API. Various data accessed and generated in connection with the provider portal (e.g., data relating to the editing of prescribed medications) can be stored in the personal health record of the monitored user. Via the above-discussed synchronization between the personal health record and one or more external electronic health records, the data can be added to one or more external electronic health records. In this way, as just an example, changes in prescribed medications or other aspects of the care plan for the monitored user can be applied (e.g., singularly or in batches) utilizing such synchronization. Moreover, the provider portal can be connected with electronic health record data, such that it exchanges insights with care team members through an existing electronic health record system, through its own user interface, or that of the integrated records database.

Further, in various embodiments the system can, in connection with the forums, warn (e.g., via the mobile app) individuals using the forums that: a) information posted in the forums should not be considered medical advice; b) individuals using the forums should contact their own care team members (e.g., their own physicians) for medical advice; and/or c) the forums do not involve (or do not necessarily involve) care team member-patient (e.g., physician-patient) relationships, or may set forth one or more legal disclaimers. Further still, in various embodiments: a) access to the forums can be limited to users of the system; b) individuals accessing the forums can be subjected to authentication by the system; and/or c) access to the forums can be limited to those individuals invited by the system. Also, in various embodiments either or both of the community portal and the provider portal can interface with one or more external social networks (e.g., Facebook, Instagram, and/or LinkedIn). Such interface can allow, for example, for posts to be shared between the portals and the one or more external social networks.

### Games/Entertainment Operations

The system can perform operations including providing health, fitness, and wellness games (e.g., brain health, exercise, and/or dexterity games). In various embodiments, the system can provide such functionality via the games/entertainment module 119. The provided games can be accessible via the virtual assistant capability, via the mobile app, and/or via IoT devices, as just some examples.

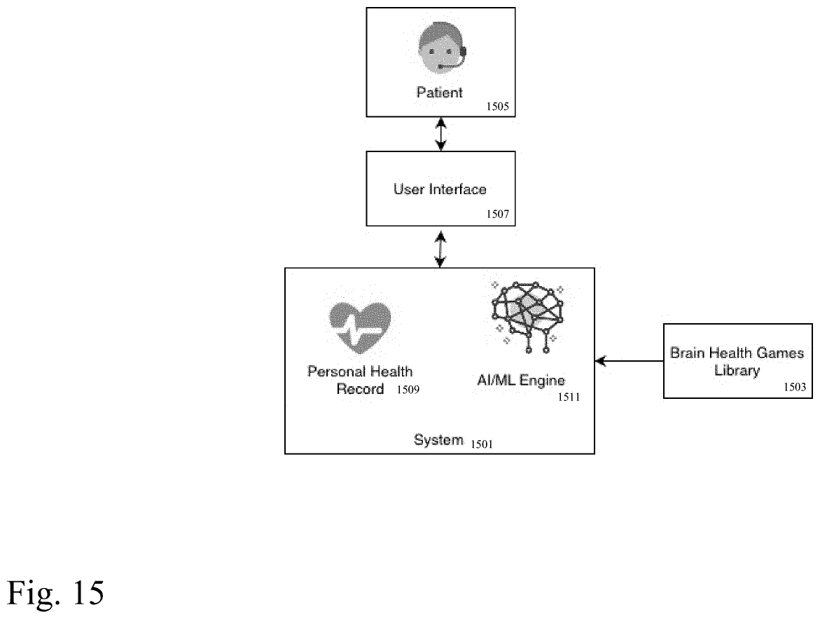

With reference to FIG. 15, the system 1501 can host a library 1503 of health, fitness, and wellness games (labeled “Brain Health Games Library” in FIG. 15). The games can be audio and/or touch based. The system can serve the games to the monitored user 1505 (labeled “Patient” in FIG. 15) through the virtual assistant capability and/or via the mobile app 1507 (labeled “User Interface” in FIG. 15), as appropriate. Further, the system can record the interactions of the monitored user with the games in various formats (e.g., audio, video, or text format). The system can also make record of scores/milestones achieved by the monitored user in the games. Also, in various embodiments physiological responses (e.g., heart rate and ECG) can be captured from the monitored user during gameplay, such as via one or more IoT devices. Such recorded interactions, record of scores/milestones achieved, and physiological response data can be stored by the system (e.g., via the storage module 127). As just one example, such data can be stored in the personal health record 1509 of the monitored user. The system can also utilize such data as inputs to one or more MLMs of the system 1511 (labeled “AI/ML Engine” in FIG. 15). In this way, the system can receive from such MLMs various useful outputs regarding the monitored user, such as care recommendations, and/or predicted conditions/health status. The system can share such MLM outputs with consent-approved parties (e.g., with care team members and family members approved by the monitored user). The system can also allow the monitored user to play health and fitness games to create greater adherence to the care plan, such as exercises for diabetes management. In ways such as this, the games/entertainment module 119 can act to provide educational functionality.

### Registration, Billing and Administrative Operations

The system can perform operations including registering a new monitored user with the system and handling billing of fees incurred through usage of the system. Further, the system can perform operations including allowing for the selection/viewing of various settings relating to usage of the system. In various embodiments, the system can provide such functionality via the registration/billing/settings module 123. Also, the system can perform operations including allowing system administrators to perform various administrative tasks relating to the system. In various embodiments, the system can provide such functionality via the administration module 125.

Turning to registration of a new monitored user with the system, a web and/or mobile app-based registration process can capture both health-related information and non-health related information (e.g., name and address) about the new monitored user. The captured health-related information can include keywords generated by the webpage or mobile app in response to the new monitored user answering various posed health questions. The registration process can also include obtaining billing information regarding the new monitored user. Information received during the registration of the new monitored user can, as just one example, be stored in the personal health record of the monitored user. Further, in various embodiments, information received during registration (e.g., health-related information) can be used to categorize the monitored user into one or more categories. As just some examples, such categories can include a diabetic category, a limited mobility category, a post-stroke category, and an epileptic category. The categorization can be performed by the system, for example, via a MLP-based classifier which has been trained to take health-related information (e.g., in the form of keywords) as input, and generate as output a category of the sort noted. The system can utilize the one or more categories to which the new monitored user is assigned for various purposes. Such purposes include condition-specific monitoring (e.g., monitoring for loss of consciousness where the monitored user has been assigned to the epileptic category) and condition-specific features (e.g., tracking blood sugar levels where the monitored user has been assigned to the diabetic category), as just some examples.

In registering the new monitored user with the system, the system can authenticate the monitored user to ensure that they are who they claim to be. As just an example, the system can utilize the ID.me API in doing so. Alternately or additionally, the system can have the monitored user provide (e.g., via smartphone camera image) a credit card and/or driver's license for authentication purposes. Further, the system can authenticate the monitored user during subsequent logins (e.g., via two-factor authentication and/or biometrics). Moreover, in various embodiments the system can continually authenticate the monitored user, such as continually during a given session between the monitored user and the system. As just one example, biometrics (voice/speech patterns of the monitored user) can be used to perform such continual authentication. Via the noted functionality, the system can help protect data of the monitored user in a HIPAA-compliant way, as just one example. Once the new monitored user has been registered with the system, this system can pass user account data back to the monitored user (e.g., the monitored user can be informed of their username and password). A new care team member or a new family member can be registered with the system in a manner analogous to that discussed in connection with registration of a new monitored user. It is noted that, in general, all users of the system (e.g., monitored users, family members, and care team members) are to register with the system prior to their usage of the system. It is further noted that a family member or a care team member can register with the system prior to or subsequent to registration of a corresponding monitored user.

Regarding billing, the system can bill customers through a web or mobile app interface, as just some examples. In various embodiments, external billing solutions can be utilized for added functionality. Billing can be done via one of multiple subscription models, of which some subset will be available depending on the partnership or customer type. The subscription models can include individual subscriptions, healthcare partnerships, and corporate partnerships.

For an individual subscription, subscription can be funded through a credit card (or other payment source) provided by the monitored user, a family member, or other individual. The payment source can be billed on a recurring basis for the services provided by the system. For healthcare partnership-oriented billing, a subscription to services provided by the system can be funded through a partnership with a healthcare provider, employer, or insurance provider. Here, a subscription to the services provided by the system can be granted with verification that the monitored user possesses coverage benefits with the healthcare or insurance provider. For corporate partnership-oriented billing, a subscription to services provided by the system can be funded through a partnership with a business of any size (e.g., anywhere from a large enterprise to a small business). Here, a subscription to the services provided by the system can be granted with verification of coverage. It is noted that, in various embodiments, the system can directly bill a Health Savings Account (HSA). As just some examples, such an HSA can be the HSA of the monitored user or the HSA of a family member of the monitored user.

Also, in various embodiments the system can provide (e.g., via the app or virtual assistant capability) estimated costs regarding healthcare usage. As just an example, the system can utilize a Long Short-Term Memory (LSTM)-based MLM provided by the machine learning module 129 to estimate such costs based on historical data. Such estimated healthcare usage costs can include costs incurred in using the system. Such estimated healthcare usage costs can also include costs incurred external to the system (e.g., prescription and physician visit costs), where the system has access to corresponding historical information. In this way, benefits such as aiding in healthcare price transparency can accrue.

Turning to allowing for the selection/viewing of various settings, the system can allow for, via the mobile app or the virtual assistant capability, the setting of application settings and monitored user/patient settings. The application settings can enable a monitored user/family member/care team member user to choose various application settings such as various preferences (e.g., reminder frequencies, interface colors, interface sounds, screen layouts, and virtual assistant voice preferences). The system can use the storage module 127 to store the application settings of a given user in a user settings data structure (or database) for that user, as just one example. Then, the patient settings can enable a monitored user/family member/care team member user to manage health data, such as but not limited to diagnosed conditions, prescribed medications, when medications are taken, health-related appointments, and other patient health data.

Turning to administrative tasks, the system can offer (e.g., via a webpage or a mobile app) an administrative panel which can allow administrators of the system to perform various tasks. As just some examples, these tasks can include: a) searching for users of the system (e.g., monitored users, family members, and care team members); b) editing user-entered registration data; c) editing registration plans for users; d) viewing holistic patent health data for monitored users (e.g., in a way compliant with HIPAA or other relevant regulations); e) viewing and editing calendar data and medication reminders for monitored users of the system; f) viewing usage data; g) creating a new user or plan; h) deleting a user or plan; i) viewing various analytics; j) integrating (e.g., via API) the system with third-party devices, services, electronic health records, and/or organizations. The viewable analytics can include number of users, number of users according to type (e.g., monitored user, family member, and care team member), number of conversations between monitored users and the virtual assistant capability, number of data points (amount of data collected for purposes of MLM input and/or amount of data generated as MLM output), frequency of engagement, and system operational performance, as just some examples. The viewable analytics can generated via the analytics/data access module 117.

### Analytics and Advertising Operations

The system can perform operations including generating data reports. In various embodiments, the system can provide such functionality via the analytics/data access module 117. Also, the system can perform operations including selecting advertisements to be displayed to the monitored user. In various embodiments, the system can provide such functionality via the advertisements module 121.

Turning to generating data reports, the generated data reports can include summary displays which regard the monitored user. The summary displays can be viewed (e.g., via the mobile app) by the monitored user, family members, and care team members. In some embodiments, the system can allow for viewing of the data reports via the provider portal.

As just some examples, the summary displays can convey data collected from electronic health records, data collected from IoT/health-monitoring devices, data regarding support services, outputs generated by MLMs of the system, indication of the extent to which medications and medical appointments are not forgotten, and indication of care recommendations made by the system. As such, in generating the data reports, the system can acquire data about the monitored user from various sources, including but not limited to the patient hub, the care team member hub, IoT/health-monitoring device data, and the MLMs of the system. Also, the analytics/data access module 117 can be used to generate the various analytics discussed above in connection with administrative tasks. As referenced above, the analytics/data access module 117 can generate analytics including number of users, number of users according to type, number of conversations between monitored users and the virtual assistant capability, number of data points, frequency of engagement, and system operational performance, as just some examples. Moreover, in various embodiments the analytics/data access module 117 can generate analytics that show that the system provides, to the monitored user, improvement in real-world benefits (e.g., quality of life, faster return to work, and/or ability to be active). In this way the system can, as just one example, provide appropriate evidence to payers that implement value-based payment schemes.

As to selecting advertisements, the system can select, based on information possessed by the system, advertisements regarding devices and services potentially useful to the monitored user. In selecting the advertisements, factors taken into account can include medical conditions, symptoms, location, health data, demographics, and prior behavior, as just some examples. In various embodiments, one or more recommender MLMs possessed by the system can be used in such selection. Alternately or additionally, the advertisements can be selected via third-party analytics software or via a third-party analytics webservice (e.g., accessed via the system by an API of the webservice). The selected advertisements can be presented to the monitored user via the mobile app or the virtual assistant capability. In various embodiments, the system can allow the monitored user to specify (e.g., via the app or virtual assistant capability) a desire to opt-out of such personalized advertising. Where the monitored user so specifies, the monitored user can receive non-personalized advertisements or be invited to pay a higher fee to utilize the system without being shown advertisements, as just some examples.

### EXAMPLES

Using the system functionality discussed herein the monitored user, care team members, and family members can perform various operations. Various examples of such operations will now be discussed.

As an example, a care team member or a family member can be a user of the system, and a monitored user can also be a user of the system. Further according to the example, the care team member or family member can desire that the system establish linkage with the monitored user. Such a circumstance can arise, for example, where the monitored user first becomes a patient of the care team member. Such a circumstance can also arise, as another example, where a family member becomes a user of the system at a point in time at which the monitored user is already a user of the system. The care team member or family member can indicate the linkage desire to the system via the mobile app or via the virtual assistant capability. In response the system can generate a Uniform Resource Locator (URL) link that the care team member or family member can provide (e.g., via messaging or email) to the monitored user). In response to the monitored user clicking on the link, the system can (e.g., via the storage module 127) establish a linkage within the system between the care team member or family member and the monitored user. The linkage can, in various embodiments, grant various rights within the system to the care team member or family member (e.g., the right to view medical information regarding the monitored user). As a further example, a care team member or family member can desire to add an individual as a new monitored user within the system. Such a circumstance can arise, for example, where the care team member gains a new patient, and the patient is not already a user of the system. Such a circumstance can also arise, as another example, where the family member desires that a person with whom they are related (e.g., a mother, a father, an aunt, or a uncle of the family member) become a user of the system. Here, the system can create a new user account for the monitored user, and subsequently perform the discussed operations to establish linkage with the monitored user.

As another example, a care team member or family member can desire to view a health report regarding a monitored user with whom they are linked in the system. Here, health data procured through various sources (e.g., through IoT device APIs) can, as just one example, be consolidated by the system and shared through HIPAA-compliant methods with the care team member or family member in a visual format via the mobile app. As a further example, a care team member can desire to schedule an appointment for a monitored user with whom they are linked in the system. Here, the care team member can interact with the system through the virtual assistant capability or the mobile app, and indicate to the system a desire to schedule a new appointment. The care team member can provide the details of the appointment to the system, such as the title of the appointment, the day, time, the doctor (or other care team member), address, phone number, and/or other notes. In response, the system can perform operations including using the alerts/notifications/reminders module 105 to add the appointment to the calendar of the monitored user. As another example where a care team member desires to schedule a new appointment for such a monitored user, the following can occur. The care team member can schedule the appointment via an electronic health record of a facility (e.g., hospital or clinic) with which they are associated. Subsequently, the care team member can (e.g., via the app or virtual assistant capability) instruct the system to access the electronic health record to receive the details of the appointment. The system can subsequently use the data acquisition/link module 103 and a medical record API to retrieve the appointment details. The system can then add the appointment to the calendar of the monitored user as discussed.

As an additional example, a care team member or family member can desire to schedule a new medication for a monitored user with whom they are linked in the system. Here, the care team member or family member can interact with the system through the virtual assistant capability or the mobile app to specify the type of medication and how frequently the monitored user takes it. In response, the system can use the alerts/notifications/reminders module 105 to accordingly populate the calendar of the monitored user. As another example where the user who desires to schedule a new medication is, in particular, a care team member, the following can occur. The care team member can schedule the medication via an electronic health record of a facility with which they are associated. The care team member can then instruct the system to access the electronic health record to receive the details of the new medication scheduling. The system can use the data acquisition/link module 104 and a medical record API to receive the details. The system can then populate the calendar of the monitored user as discussed. Also as an example, a care team member or family member can desire to view the schedule of a monitored user with whom they are linked in the system. Here, the system can (e.g., via the mobile app) allow the care team member or family member to navigate to and view the calendar of the monitored user.

As another example, a care team member can desire to call another care team member (e.g., a physician) of a monitored user with whom they are linked in the system. Here, the system can, via the mobile app or virtual assistant capability, allow the care team member to select the desired target care team member from the care directory. The care team member can then use the app or virtual assistant capability to indicate/confirm a desire to call the selected target. In response, the system can (e.g., via the care coordination module 111) connect the care team member to the target, using the corresponding telephone number stored in the care directory. As a further example, a care team member, family member, or monitored user can desire to change their application settings. Here, the care team member, family member, or monitored user can use the mobile app or virtual assistant capability to specify the desired settings changes. In response, the system can make corresponding changes in the user settings data structure (or database) for that the care team member, family member, or monitored user. As yet another example, a care team member or family member can desire to change monitored user/patient settings for a monitored user with whom they are linked in the system. Here, the care team member or family member can use the mobile app or the virtual assistant capability to indicate the desired changes (e.g., changes to diagnosed conditions and/or prescribed medications). In reply, the system can instantiate the changes. As a further example where a care team member desires to change monitored user/patient settings (e.g., changes to diagnosed conditions and/or prescribed medications) for such a monitored user, the following can occur. The care team member can make the desired changes via an electronic health record of a facility with which they are associated. Subsequently, the care team member can instruct the system to access the electronic health record to receive the details of the changes. The system can then, via the data acquisition/link module 103 and a medical record API, access the electronic health record and retrieve the details. The system can then instantiate the changes.

As an additional example, a care team member or family member can desire to view a transcript of a particular conversation (e.g., the most recent conversation) between the virtual assistant capability and a monitored user with whom that care team member or family member is linked in the system. Here, the care team member or family member can indicate such desire via the virtual assistant capability or the mobile app. As just an example, where the mobile app is used the care team member or family member can use a UI of the mobile app to select the desired conversation (e.g., the most recent conversation). In reply, the system can use the mobile app to present to the care team member or family member a textual representation of the transcript, or use the virtual assistant capability to speak the transcript, using the voice of the virtual assistant. Also as an example, a care team member or family member can desire to utilize the forums. Here, the care team member can use the mobile app to navigate to the provider portal. Likewise, the family member can use the mobile app to navigate to the community portal. Within the selected portal, the care team member or family member can perform operations including selecting a topic or channel of interest, viewing postings/links thereof, and “liking” a comment. Further, the care team member or family member can post comments. As an illustration, the family member can engage with the community, post about tips and tricks, and see what has worked well for members of other families.

As another example, the monitored user can desire to know when to take their medications. Here, the monitored user can use the mobile app or the virtual assistant capability to request that the system inform them of the next medication reminder. The system can then use the alerts/notifications/reminders module 105 to access the calendar for the monitored user, and to determine the next-due medication reminder. Subsequently, the system can use the app or virtual assistant capability to convey that reminder to the monitored user. As a further example, the monitored user can desire to call a care team member or a family member. Likewise, a family member can desire to call a care team member, the monitored user, or another family member. Here, the system can, via the mobile app or virtual assistant capability, allow the monitored user or the family member to select the desired target user (e.g., care team member or family member) from the care directory. The monitored user or family member can then use the app or virtual assistant capability to indicate/confirm a desire to call the selected target. In response, the system can (e.g., via the care coordination module 111) connect the monitored user or family member to the target, using the corresponding telephone number stored in the care directory. As an additional example, a family member can desire to add a new appointment to the schedule of the monitored user, or the monitored user can desire to add a new appointment to their own schedule. Here, the family member can be linked in the system to the monitored user. According to this example, the monitored user or family member can interact with the system through the virtual assistant capability, the web app, or the mobile app to provide the details of the appointment (e.g., title of the appointment, the day, time, the doctor (or other care team member), address, phone number, and/or other notes). Alternatively, the system can determine the details of the appointment via a transcribed call. Here, the monitored user or family member can use the system to call the care team member with whom the appointment is to be made, as discussed above. The system can record and transcribe the telephone call, and extract therefrom details regarding an appointment made during the call. In this regard, the system can utilize an RNN-based MLM of the machine learning module 129 that has been trained to identify appointment-related verbiage from a block of text. Using the details of the appointment, the system employ use the alerts/notifications/reminders module 105 to accordingly populate the calendar of the monitored user. Also as an example, the monitored user can desire to view their appointments. Here, the system can, via the mobile app or virtual assistant capability, present the appointments to the monitored user.

As another example, the monitored user can desire to change their own monitored user/patient settings. Here, the monitored user can use the mobile app, the web app, or the virtual assistant capability to indicate the desired changes (e.g., changes to diagnosed conditions and/or prescribed medications). In reply, the system can instantiate the changes. As a further example, the monitored user can desire to purchase a third-party support service (e.g., a laundry service). Likewise, a family member can desire to purchase a third-party support service on behalf of a monitored user with whom they are linked in the system. Here, the system can, via the mobile app or the virtual assistant capability, allow the monitored user or family member to browse or search for available services, and to view details regarding available services (e.g., prices and telephone numbers). The app or virtual assistant capability can allow the monitored user or family member to select a service that they desire, and to specify relevant details (e.g., desired laundry pick-up time). As one example, the system can (e.g., via the API integrations module) connect with the selected third-party service via an API thereof to secure the desired service. As another example, the system can transfer the monitored user or family member to an external system to perform the ordering process (e.g., the mobile app can open a browser window to a website of the third-party support service). As just an example, billing can be done independently by each service provider. In various embodiments, the monitored user can specify that a family member handle bill payment responsibilities for purchased third-party support services,

### Hardware and Software

According to various embodiments, various functionality discussed herein can be performed by and/or with the help of one or more computers. Such a computer can be and/or incorporate, as just some examples, a personal computer, a server, a smartphone, a system-on-a-chip, and/or a microcontroller. Such a computer can, in various embodiments, run Linux, MacOS, Windows, or another operating system.

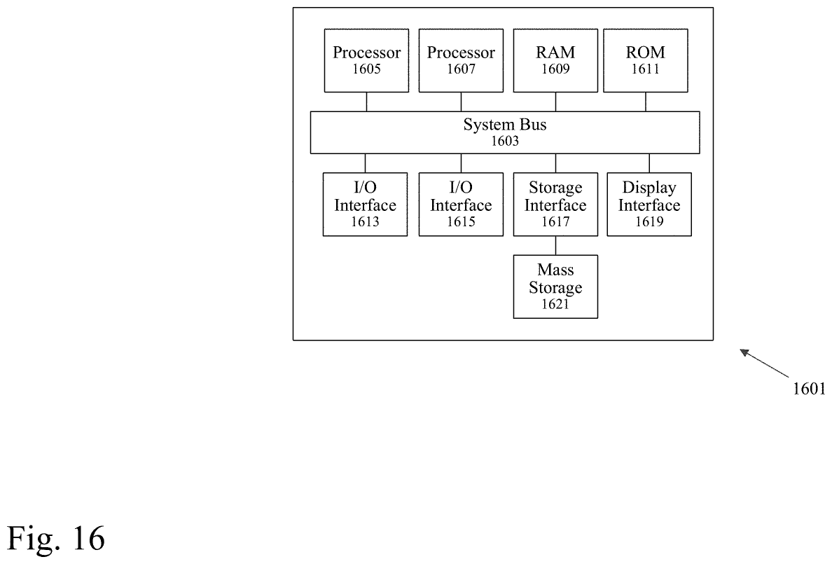

Such a computer can also be and/or incorporate one or more processors operatively connected to one or more memory or storage units, wherein the memory or storage may contain data, algorithms, and/or program code, and the processor or processors may execute the program code and/or manipulate the program code, data, and/or algorithms. Shown in FIG. 16 is an example computer employable in various embodiments of the present invention. Exemplary computer 1601 includes system bus 1603 which operatively connects two processors 1605 and 1607, random access memory (RAM) 1609, read-only memory (ROM) 1611, input output (I/O) interfaces 1613 and 1615, storage interface 1617, and display interface 1619. Storage interface 1617 in turn connects to mass storage 1621. Each of I/O interfaces 1613 and 1615 can, as just some examples, be a Universal Serial Bus (USB), a Thunderbolt, an Ethernet, a Bluetooth, a Long Term Evolution (LTE), a 5G, an IEEE 488, and/or other interface. Mass storage 1621 can be a flash drive, a hard drive, an optical drive, or a memory chip, as just some possibilities. Processors 1605 and 1607 can each be, as just some examples, a commonly known processor such as an ARM-based or x86-based processor. Computer 1601 can, in various embodiments, include or be connected to a touch screen, a mouse, and/or a keyboard. Computer 1601 can additionally include or be attached to card readers, DVD drives, floppy disk drives, hard drives, memory cards, ROM, and/or the like whereby media containing program code (e.g., for performing various operations and/or the like described herein) may be inserted for the purpose of loading the code onto the computer.

In accordance with various embodiments of the present invention, a computer may run one or more software modules designed to perform one or more of the above-described operations. Such modules can, for example, be programmed using Python, Java, JavaScript, Swift, React, C, C++, C#, and/or another language. Corresponding program code can be placed on media such as, for example, DVD, CD-ROM, memory card, and/or floppy disk. It is noted that any indicated division of operations among particular software modules is for purposes of illustration, and that alternate divisions of operation may be employed. Accordingly, any operations indicated as being performed by one software module can instead be performed by a plurality of software modules. Similarly, any operations indicated as being performed by a plurality of modules can instead be performed by a single module. It is noted that operations indicated as being performed by a particular computer can instead be performed by a plurality of computers. It is further noted that, in various embodiments, peer-to-peer and/or grid computing techniques may be employed. It is additionally noted that, in various embodiments, remote communication among software modules may occur. Such remote communication can, for example, involve JavaScript Object Notation-Remote Procedure Call (JSON-RPC), Simple Object Access Protocol (SOAP), Java Messaging Service (JMS), Remote Method Invocation (RMI), Remote Procedure Call (RPC), sockets, and/or pipes.

Moreover, in various embodiments the functionality discussed herein can be implemented using special-purpose circuitry, such as via one or more integrated circuits, Application Specific Integrated Circuits (ASICs), or Field Programmable Gate Arrays (FPGAs). A Hardware Description Language (HDL) can, in various embodiments, be employed in instantiating the functionality discussed herein. Such an HDL can, as just some examples, be Verilog or Very High Speed Integrated Circuit Hardware Description Language (VHDL). More generally, various embodiments can be implemented using hardwired circuitry without or without software instructions. As such, the functionality discussed herein is limited neither to any specific combination of hardware circuitry and software, nor to any particular source for the instructions executed by the data processing system.

### Ramifications and Scope

Although the description above contains many specifics, these are merely provided to illustrate the invention and should not be construed as limitations of the invention's scope. Thus, it will be apparent to those skilled in the art that various modifications and variations can be made in the system and processes of the present invention without departing from the spirit or scope of the invention.

In addition, the embodiments, features, methods, systems, and details of the invention that are described above in the application may be combined separately or in any combination to create or describe new embodiments of the invention.

1. A computer-implemented method, comprising:
providing, by a computing system, to a machine learning model, input data for a monitored user, wherein the input data comprises verbal-based data;
receiving, by the computing system, from the machine learning model, generated output, wherein the generated output comprises at least one of a condition/health status or a care recommendation; and
communicating, by the computing system, using one or more of a mobile app or a virtual assistant capability, one or more of the condition/health status or the care recommendation.

2. The computer-implemented method of claim 1, wherein the input data further comprises at least one of data regarding internet of things (IoT)/health-monitoring device outputs or data drawn from electronic health records.

3. The computer-implemented method of claim 1, wherein the verbal-based data comprises at least one of data corresponding to verbal inputs provided by the monitored user to the virtual assistant capability, or data drawn from communications between the monitored user and at least one of family members or care team members.

4. The computer-implemented method of claim 1, wherein the verbal-based data comprises keywords generated by the computing system from at least one of verbal inputs provided by the monitored user to the virtual assistant capability, or communications between the monitored user and at least one of family members or care team members.

5. The computer-implemented method of claim 1, wherein the input data further comprises data corresponding to a registration of the monitored user with the computing system.

6. The computer-implemented method of claim 1, wherein the generated output further comprises an urgency indicator.

7. A computer-implemented method, comprising:
determining, by a computing system, that a communication is to be executed for a monitored user, wherein the determination utilizes one or more of IoT/health-monitoring device output or calendar functionality;
providing, by the computing system, to a machine learning model, input data for the monitored user;
receiving, by the computing system, from the machine learning model, generated output, wherein the generated output comprises at least one of a medical profession type or a physician type;
determining, by the computing system, at least one care team member who matches said generated output of the machine learning model; and
executing, by the computing system, the communication for the monitored user, wherein the communication is between the monitored user and said at least one determined care team member.

8. The computer-implemented method of claim 7, wherein the communication is one of a call, a text chat, an audio chat, or a video chat.

9. The computer-implemented method of claim 7, wherein the determination that the communication is to be executed utilizes said IoT/health-monitoring device output, and wherein the determination comprises at least one of ascertaining that the monitored user has fallen, ascertaining that the monitored user has suffered a cardiac arrest, ascertaining that the monitored user has suffered a stroke, ascertaining that the monitored user has suffered loss of consciousness, or ascertaining that the monitored user has suffered an asthma attack.

10. The computer-implemented method of claim 9, further comprising:
executing, by the computing system, communication between the monitored user and at least one family member.

11. The computer-implemented method of claim 7, wherein the determination that the communication is to be executed utilizes the calendar functionality, and wherein the determination comprises ascertaining that the monitored user has at least one of an upcoming health appointment or an upcoming wellness appointment.

12. The computer-implemented method of claim 7, wherein the determination of the at least one care team member comprises consulting a care directory.

13. The computer-implemented method of claim 7, wherein the input data comprises at least one of verbal-based data, data regarding IoT/health-monitoring device outputs, or data drawn from electronic health records.

14. A computer-implemented method, comprising:
generating, by a computing system, for a monitored user, at least one of health-related alerts, health-related notifications, or health-related reminders;
receiving, by the computing system, at least one of electronic health record data of the monitored user or IoT/health-monitoring device output for the monitored user;
generating, by the computing system, using at least one machine learning model, at least one of condition/health statuses or care recommendations for the monitored user; and
storing, by the computing system, in a personal health record of the monitored user, at least one of the electronic health record data, the IoT/health-monitoring device output, the condition/health statuses, or the care recommendations.

15. The computer-implemented method of claim 14, wherein said at least one of health-related alerts, health-related notifications, or health-related reminders are, with the consent of the monitored user, shared with at least one of care team members or family members.

16. The computer-implemented method of claim 14, wherein said at least one of health-related alerts, health-related notifications, or health-related reminders regard at least one of emergent situations, care recommendations, care coordination reports, prescription refill statuses, medication dosings, or upcoming health appointments.

17. The computer-implemented method of claim 14, further comprising:
implementing, by the computing system, communications between the monitored user and at least one of care team members or family members, wherein the communications comprise at least one of calls, text chats, audio chats, video chats, or forums; and
storing, by the computing system, in the personal health record of the monitored user, data regarding the communications.

18. The computer-implemented method of claim 14, further comprising:
acquiring, by the computing, for the monitored user, one or more support services, wherein the computing system connects with the one or more support services via at least one of Application Programming Interface (API), screen scraping, or portal.

19. The computer-implemented method of claim 14, further comprising:
providing, by the computing system, to the monitored user, at least one of health, fitness, or wellness games; and
storing, by the computing system, in the personal health record, data regarding interaction of the monitored user with said at least one of health, fitness, or wellness games.

20. The computer-implemented method of claim 19, further comprising:
recommending, by the computing system, utilizing at least one machine learning model, at least one of a health game, a fitness game, or a wellness game.
